(19) United States
(12) Patent Application Publication
(10) Pub. No.: US 2019/0054035 A1
(43) Pub. Date: Feb. 21, 2019

(54) SELF-ASSEMBLED PEPTIDE NANOFIBERS AND USE THEREOF FOR TARGETED DRUG DELIVERY

(71) Applicants: Yissum Research Development Company of the Hebrew University of Jerusalem Ltd., Jerusalem (IL); Ramot at Tel-Aviv University Ltd., Tel Aviv (IL)

(72) Inventors: Galia BLUM, Maccabim (IL); Yael BEN-NUN, Ramat Gan (IL); Ehud GAZIT, Ramat Hasharon (IL); Lihi ADLER-ABRAMOVICH, Herzeliya (IL); Galit FICHMAN, Yavne (IL)

(73) Assignees: Yissum Research Development Company of the Hebrew University of Jerusalem Ltd., Jerusalem (IL); Ramot at Tel-Aviv University Ltd., Tel Aviv (IL)

(21) Appl. No.: 15/949,484

(22) Filed: Apr. 10, 2018

Related U.S. Application Data
(60) Provisional application No. 62/483,553, filed on Apr. 10, 2017.

(51) Int. Cl. A61K 9/70; A61K 47/42; A61K 31/704

(52) U.S. Cl. CPC A61K 9/70; A61K 31/704; A61K 47/42

(57) ABSTRACT

Provided are peptide-based nanostructures, as well as diagnostic and therapeutic methods using same.

### TECHNOLOGICAL FIELD

The present disclosure relates to self-assembled peptides and peptide nanostructures for use in drug delivery.

### BACKGROUND

Short peptide sequences for nanotechnological and nanomedicinal applications have gained popularity due to their biodegradability, simple preparation, reproducibility and ability to spontaneously form 3D structures. One of the studied building blocks are the Phe-Phe (FF) variants which self-assemble into nanostructures such as tubular or spherical nanostructures via hydrophobic and aromatic π-π stacking interactions [1-3]. Self-assembly of Phe-Phe (FF) variants into nanofibers was possible upon modification with aromatic end capping moieties [3].

Enzymatic degradation of self-assembled Phe-Phe (FF) nanotubes was previously detected [4].

Self-assembled nanofibers of D-peptides with 10-hydroxycamptothecin were reported to have better selectivity and antitumor activity than nanofibers of L-peptides, suggesting the importance of peptidase resistance D-fiber systems as vehicles of drugs in therapy [5].

Dendrimer peptides with tetra-peptide spacers (Gly-Phe-Leu-Gly) and anticancer drugs were shown to be suitable substrates for protease cathepsin B [6].

### REFERENCES

[1] International Patent Publication WO04052773.
[2] International Patent Publication WO04060791.
[3] International Patent Publication WO06027780.
[4] Reches M and Gazit E. Science 2003, 300, 625-627.
[5] Liu, J.; Liu, J.; Chu, L.; Zhang, Y.; Xu, H.; Kong, D.; Yang, Z.; Yang, C.; Ding, D. ACS Appl. Mater. Interfaces 2014, 6, 5558-5565.
[6] Li N.; Li N.; Yi Q.; Luo K, Guo C.; Pan D.; Gu Z. Biomaterials. 2014, 35, 9529.

### SUMMARY OF THE INVENTION

The invention disclosed herein is based on the development of peptides that, on one hand, are selectively recognized and cleaved by proteases, such as cathepsin, and, on the other hand, self-assemble into nanostructures, such as nanofibers. While self-assembly into nanostructures of aromatic short peptides is known, the inventors have surprisingly found that self-assembly of peptides containing hydrophilic moieties is not hampered or otherwise prevented due to the presence of the hydrophilic moieties that are part of the protease recognition and cleavable sequence (region). Furthermore, the inventors have found that a protease is capable of selectively accessing and cleaving the peptide in the assembled nanostructure.

Also, nanostructures of the invention can be used for a variety of applications, such as for selective delivery of agents in medical and biological applications, e.g., for releasing an agent of interest, such as a drug, at sites of high protease presence and activity. Development of such specific delivery systems or of selective release systems is highly advantageous as it permits delivery of high drug concentrations to target sites and low drug concentrations to non-target sites, thereby decreasing side effects and drug toxicity while maximizing efficacy.

In accordance with aspects of the invention, a nanostructure is provided that comprises a plurality of peptides, wherein at least a portion of the plurality of peptides comprises peptides having a pi-pi (π-π) interacting moiety and at least one protease interacting moiety, wherein the π-π interacting moiety and the at least one protease interacting moiety are different, and wherein the at least one protease interacting moiety having at least one protease cleavable bond. The nanostructure may be free of dipeptides (peptides structured of two amino acids).

Thus, the invention provides a nanostructure comprising a plurality of peptides, none of said peptides being a dipeptide, wherein at least a portion of the plurality of peptides comprises peptides having a pi-pi (π-π) interacting moiety and at least one protease interacting moiety, wherein the π-π interacting moiety and the at least one protease interacting moiety are different, and wherein the at least one protease interacting moiety having at least one protease cleavable bond.

The invention further provides a cathepsin cleavable nanostructure, the nanostructure comprising a plurality of peptides, wherein at least a portion of the plurality of peptides is selected to undergo cleavage by cathepsin, the at least portion comprises peptides having a pi-pi (π-π) interacting moiety and at least one protease interacting moiety, wherein the π-π interacting moiety and the at least one protease interacting moiety are different, and wherein the at least one protease interacting moiety having at least one cathepsin cleavable bond.

The invention further provides a cathepsin cleavable nanostructure comprised of a plurality of cathepsin cleavable peptides, the peptides comprising each a pi-pi (π-π) interacting moiety and at least one protease interacting moiety, wherein the π-π interacting moiety and the at least one cathepsin interacting moiety are different, and wherein the at least one cathepsin interacting moiety having at least one cathepsin cleavable bond.

The “nanostructure” of the invention, constructed of peptides, as defined herein, is an ordered arrangement having at least one of its dimensions in the nanometric range. Non-limiting examples of nanostructures include a nanoparticle, a nanotube, a nanofiber, a nanowire and a nanorod. The nanostructure of the present invention can have any shape, e.g., planar, spherical, tubular or fibrillary. In some embodiments, the nanostructure is a nanofiber. In accordance with some aspects, the present disclosure provides a nanofiber comprising at least one peptide, as detailed herein. Similarly, a “nanofiber”, also denoted fibrillar nanostructure, is a nanostructure in the form of a filament or a fiber having a diameter or a cross section of less than 1 micrometer. In some embodiments, the nanofiber has a diameter or a cross section of less than 500 nm. In some embodiments, the diameter or cross section of the nanofiber is between about 10 nm and about 320 nm, between about 20 nm and about 150 nm, between about 20 nm and about 100 nm or between about 20 nm to about 30 nm. The length of the nanofiber is on average over 10 micrometer.

The nanostructures, e.g., nanofibers, of the invention are constructed of a plurality of “peptide” molecules, each peptide having between 3 and 10 amino acids. In some embodiments, the number of amino acids is between 3 and 6, between 4 and 6, between 3 and 5, or 3 or 4 or 5 or 6 or 7 or 8 or 9 or 10 amino acids. The peptides are not dipeptides, nor homo-dipeptides.

In some embodiments, the peptides comprise 3 or 4 amino acids as further defined herein, each of the amino acids associated to another amino acid optionally via a peptide bond. The peptide may be selected from synthetically prepared, recombinant or degraded products, and peptidomimetics as well as peptoids and semipeptoids.

In some embodiments, the peptide comprises at least 3 amino acids, thus having an amino acid sequence of the general formula X₀-X₂₍ₘ₎-X₃₍ₚ₎-X_{4(q)} (SEQ ID NO:1), wherein each of X₀, X₂, X₃ and X₄ is an amino acid and each of m, p and q is an integer independently selected from 1 to 3, wherein X₀ may be absent, wherein at least one of the amino acids or a combination of two or more amino acids constitutes a protease interacting moiety and at least one amino acid or a combination of two or more amino acids constitutes a pi-pi interacting moiety.

Peptides of SEQ ID NO:1 comprise a combination of amino acids such that the amino acids making up the peptide are not all the same. For example, at each of X₂, X₃ and X₄ may be selected from aromatic amino acids and hydrophilic amino acids, such that not all of X₂, X₃ and X₄ are aromatic or hydrophilic.

In some embodiments, each of X₂ and X₄ is independently selected from an amino acid or an amino acid combination being or comprising a pi-pi interacting moiety and X₃ represents an amino acid or an amino acid combination being or comprising a protease interacting moiety.

In some embodiments, each of X₂ and X₃ is independently selected from an amino acid or an amino acid combination being or comprising a pi-pi interacting moiety and X₄ represents an amino acid or an amino acid combination being or comprising a protease interacting moiety.

In some embodiments, each of X₃ and X₄ is independently selected from an amino acid or an amino acid combination being or comprising a pi-pi interacting moiety and X₂ represents an amino acid or an amino acid combination being or comprising a protease interacting moiety.

In some embodiments, each occurrence of m, p and q is independently selected from 1 to 3.

In some embodiments, each occurrence of m, p and q is 1.

In some embodiments, m is 2, p is 1 and q is 1.

In a peptide utilized in accordance with the invention and designated herein, each “—” represents a bond, e.g., a peptide bond, or a linker group associating one amino acid (or a combination of amino acids) to another amino acid (or another combination of amino acids). Where “—” denotes a peptide bond, in some embodiments, this bond may be a protease cleavable bond. Alternatively, “—” denotes a linker group or a moiety comprising at least one atom or groups of atoms. In some embodiments, “—” denotes a group of atoms, e.g., at least one amino acid, or a combination of amino acids, e.g., a peptide moiety (comprising two or more amino acid groups). In some embodiments, “—” denotes a bond that is not cleavable by a protease and thus the cleavable bond referred to herein is any bond associated to the protease interacting moiety or formed between any two atoms within the protease interacting moiety. In other words, the cleavable bond may be a bond associating the at least one protease interacting moiety with the π-π interacting moiety, or with any other functionality or group present in the peptides, or may be a bond within the at least one protease interacting moiety that is further protease cleavable.

It should be noted that were a peptide is designated herein without the use of “—”, a covalent bond, e.g., a peptide bond, may be assumed based on known bonding rules. For example, in a peptide designated FFKF, a covalent bond should be assumed to exist between each pair of amino acids, even though the bond is not indicated.

Typically, the π-π interacting moiety and the at least one protease interacting moiety are different. Yet, in some cases, the two moieties may be the same. In other words, where the two moieties are different, they may be different in the amino acids making up the moieties, the sequence of these amino acids (N to C or C to N), the presence or absence of an atom or a group of atoms, the site of association of the atom or group of atoms etc. Where the two moieties are the same, they are said to be identical to one another. Where the two moieties are different, they may be associated to each other directly (via a bond) or via a linker group. In some cases, the two moieties, namely the pi-pi interacting moiety and the protease interacting moiety may share one or more amino acids. For example, in a peptide of an exemplary sequence X₂₍ₘ₎-X₃₍ₚ₎-X_{4(q)}, the pi-pi interacting moiety may be X₂-X₃, and the protease interacting moiety may be X₃-X₄ (the group X₃ is shared by the two groups). In another example, in a peptide of the structure FFKF, the pi-pi interacting moiety is FF and the protease interacting moiety is the whole peptide, namely FFKF. As such, the two moieties are different (despite the possible inclusion of the pi-pi interacting moiety within the protease interacting moiety). The designation of a moiety as pi-pi interacting or protease interacting is determined based on the amino acids making up the moiety and their sequence, as well as on experimental data demonstrating functionality. For purposes of all or the majority of peptides disclosed herein, the peptide segment FF is not to be considered a protease interacting moiety.

In some embodiments, the peptide is of an amino acid sequence X₁₍ₙ₎-X₂₍ₘ₎-X₃₍ₚ₎-X_{4(q)} (SEQ ID NO:2), wherein each of X₂₍ₘ₎, X₃₍ₚ₎, and X_{4(q)} is as defined herein, and X₁ is similarly an amino acid and n is an integer selected from 1 to 3.

In some embodiments, each of X₁, X₂ and X₄ is independently selected from an amino acid or an amino acid combination being or comprising a pi-pi interacting moiety and X₃ represents an amino acid or an amino acid combination being or comprising a protease interacting moiety.

In some embodiments, each of X₁, X₂ and X₃ is independently selected from an amino acid or an amino acid combination being or comprising a pi-pi interacting moiety and X₄ represents an amino acid or an amino acid combination being or comprising a protease interacting moiety.

In some embodiments, each of X₂, X₃ and X₄ is independently selected from an amino acid or an amino acid combination being or comprising a pi-pi interacting moiety and X₁ represents an amino acid or an amino acid combination being or comprising a protease interacting moiety.

In some embodiments, each of X₁, X₃ and X₄ is independently selected from an amino acid or an amino acid combination being or comprising a pi-pi interacting moiety and X₂ represents an amino acid or an amino acid combination being or comprising a protease interacting moiety.

In some embodiments, each of X₁, X₂ and X₄ is independently selected from an amino acid or an amino acid combination being or comprising a protease interacting moiety and X₃ represents an amino acid or an amino acid combination being or comprising a pi-pi interacting moiety.

In some embodiments, each of X₁, X₂ and X₃ is independently selected from an amino acid or an amino acid combination being or comprising a protease interacting moiety and X₄ represents an amino acid or an amino acid combination being or comprising a pi-pi interacting moiety.

In some embodiments, each of X₂, X₃ and X₄ is independently selected from an amino acid or an amino acid combination being or comprising a protease interacting moiety and X₁ represents an amino acid or an amino acid combination being or comprising a pi-pi interacting moiety.

In some embodiments, each of X₁, X₃ and X₄ is independently selected from an amino acid or an amino acid combination being or comprising a protease interacting moiety and X₂ represents an amino acid or an amino acid combination being or comprising a pi-pi interacting moiety.

In some embodiments, X₂ is an aromatic amino acid or a hydrophilic amino acid, repeated m times, wherein m is an integer from 1 to 3; X₃ is an aromatic amino acid or a hydrophilic amino acid, repeated p times, wherein p is an integer from 1 to 3; X₄ is an aromatic amino acid or a hydrophilic amino acid, repeated q times, wherein q is an integer from 1 to 3; wherein the peptide comprises between 3 and 9 amino acids and a combination of aromatic amino acids and hydrophilic amino acids.

In some embodiments, X₁ is an aromatic amino acid or a hydrophilic amino acid, repeated n times, wherein n is an integer from 1 to 3; X₂ is an aromatic amino acid or a hydrophilic amino acid, repeated m times, wherein m is an integer from 1 to 3; X₃ is an aromatic amino acid or a hydrophilic amino acid, repeated p times, wherein p is an integer from 1 to 3; X₄ is an aromatic amino acid or a hydrophilic amino acid, repeated q times, wherein q is an integer from 1 to 3; the peptide comprising between 4 and 12 amino acids and a combination of aromatic amino acids and hydrophilic amino acids.

In some embodiments, a peptide used in accordance with the invention comprises at least one phenylalanine (F) and at least one lysine (K). In some embodiments, X₁, X₃ and X₄ are phenylalanine (F) and X₂ is lysine (K). In some embodiments, X₁, X₂ and X₄ are phenylalanine (F) and X₃ is lysine (K).

Peptides of the invention may alternatively be described by the general formula X-Y, wherein X is the protease interacting moiety and Y is the pi-pi interacting moiety, and “—” designates a bond or a linker group or atom. Note that in the general formula X-Y, each of the letters X and Y designates a variable group, not a specific amino acid.

Without wishing to be bound by theory, it is believed that the amino acid sequence denoted X provides a specific recognition and interaction with a protease such as cathepsin, thereby resulting in a bond cleavage, wherein the cleaved bond is a cleavable bond between the two moieties or any bond to the protease interacting moiety or within the moiety. The amino acid sequence denoted Y participates in pi-pi interactions (stacking), forming the self-assembled nanostructure, e.g., nanofiber. While X and Y may be different moieties, i.e., different in length and/or selection of amino acids and/or amino acid sequence and/or selection of peptide composition and length, etc; as indicated above, the two groups may share an atom or a group of atoms or an amino acid. In other words, a peptide according to the invention may be of the form X-A-Y, wherein X-A constitutes the protease interacting moiety and A-Y constitutes the pi-pi interacting moiety. A may be an atom or a group of atoms or any amino acid.

In some cases, each X and Y may be similar or identical or different and may or may not comprise an atom or an amino acid or a peptide moiety that is shared or common to both. In other words, an amino acid may be selected to both permit or undergo interaction with the enzyme and at the same time permit or affect pi-pi interaction with another amino acid or moiety in the peptide or in the nanostructure, permitting self-assembly into the nanostructures.

The “protease interacting moiety” is an amino acid sequence that is specifically recognized by a protease and that is capable of being associated or bound by a protease (e.g. the peptide sequence which serves as a protease substrate). It should be noted that while the protease specifically interacts and may bind to the moiety sequence in the peptide, it also selectively cleaves a bond to the interacting sequence or within the interacting sequence. The protease interacting moiety may be a peptide moiety or comprising a peptide moiety selected to comprise one or more groups/atoms/bonds capable of interaction with the protease, e.g., cathepsin. In some embodiments, in a peptide of the structure X-Y, X being a protease interacting moiety comprising at least one aromatic amino acid. In some embodiments, X comprises at least one aromatic amino acid and at least one hydrophilic amino acid. In other embodiments, X comprises at least one hydrophilic amino acid and at least two aromatic amino acids. In some embodiments, X comprises alternating aromatic amino acids and hydrophilic amino acids. In some embodiments, X comprises 3 or 4 amino acids. In some embodiments, X comprises one hydrophilic amino acid (centered) between two aromatic amino acids. In some embodiments, the amino acid sequence Y comprises one or at least two amino acids, each capable of pi-pi interaction.

In a peptide of the invention, in some embodiments, in a peptide having the sequence FFKF, the protease interacting moiety (X) is the complete sequence FFKF and the pi-pi interacting moiety (Y) is FF. In some embodiments, in the peptide FFKF, the protease interacting moiety (X) is FKF and the pi-pi interacting moiety (Y) is FF. In some embodiments, the protease interacting moiety (X) is KF and the pi-pi interacting moiety (Y) is FF. in some embodiments, the protease interacting moiety is not FF.

In some embodiments, the protease interacting moiety (X) may have the amino acid sequence X₂-X₃-X₄, wherein each of X₂, X₃ and X₄ are as defined above.

In some embodiments, the protease interacting moiety (X) may have the amino acid sequence X₂-X₃-X₄ and the pi-pi interacting moiety (Y) may have the amino acid sequence X₁-X₂, wherein each of X₁, X₂, X₃ and X₄ are as defined above.

The cleavable bond, a “protease cleavable bond”, that cleaves upon interaction with the protease is typically a bond, e.g., a peptide bond, that is selectively recognized and cleaved by a protease such as cathepsin. The bond may be a bond between the pi-pi interacting moiety and the protease interacting moiety (e.g., the bond associating X to Y) or any bond to the protease interacting moiety, or any bond within the moiety. The protease may cleave one or more peptide bonds, with different selectivities. Irrespective of whether one or more bonds become eventually cleaved by the protease, at least one bond in a peptide has a greater lability to protease cleavage. Typically, this bond is not a bond within the pi-pi moiety (in other words is not a bond between F-F), but rather with or within the protease interacting moiety. As stated above, in some embodiments, the cleavable bond is a bond associating the pi-pi interacting moiety and the protease interacting moiety. At times, the protease may cleave two bonds, the first being a bond between the two moieties, as described, and a second being a bond of reduced protease affinity. In other words, the peptide may undergo protease mediated non-specific cleavage. As the peptide is tailored to specifically interact and bind the protease, it is predominantly cleaved at a protease cleavable peptide bond; namely the majority of the peptides are cleaved at a protease cleavable peptide bond and not at the pi-pi interacting moiety. This means that contrary to dipeptides, such as phenylalanine dipeptides known in the art (being excluded from the scope of the present disclosure), in which non-specific protease cleavage may occur, under certain conditions, between the two amino acids, in the peptides described herein, a specific protease cleavage occurs mainly at a protease cleavable peptide bond, for example between a lysine residue and a neighboring aromatic amino acid.

To permit selective cleavage, in some embodiments, the protease interacting moiety comprises a hydrophilic amino acid. In some embodiments, the protease interacting moiety comprises at least one hydrophilic amino acid and at least one aromatic amino acid. In some embodiments, the protease interacting moiety comprises at least one hydrophilic amino acid and at least two aromatic amino acids. In some embodiments, the protease interacting moiety comprises alternating aromatic amino acids and hydrophilic amino acids. In some embodiments, the protease interacting moiety comprises between 3 and 4 amino acids. In some embodiments, the protease interacting moiety comprises one hydrophilic amino acid optionally centered between two aromatic amino acids. In some embodiments, the protease cleavable peptide bond is a bond associating a hydrophilic amino acid and an aromatic amino acid.

In some embodiments, the protease cleavable bond is a bond (may be designated “—”) between a pair of amino acids. According to the MEROPS database (http://www.ebi.ac.uk/merops/), proteases such as Cathepsin B or Cathepsin L can recognize (interact) and cleave peptide bonds between a variety of amino acid pairs. As such, the protease cleavable bond may be between any pair of amino acid. For example, the protease cleavable bond may be a bond between the following pairs of amino acid: F-K, K-F, F-R, R-F, Y-K, K-F, Y-R, R-F, F-R and R-H, defined from the N-terminus end to the C-terminus end (each letter is the one-letter amino acid designator).

In some embodiments, the protease cleavable bond is a bond associating F to K (e.g., F-K). In some embodiments, the protease cleavable bond is a bond associating K to F (e.g., K-F). In some embodiments, the protease cleavable bond is a bond formed between F and K and/or between K and F. In some embodiments, the protease interacting moiety is selected from F-K-F, F-R-F, Y-K-F, Y-R-F and F-R-H, defined from the N-terminus end to the C-terminus end, wherein the protease cleavable bond is one or more of the designated bonds.

The “pi-pi interacting moiety” is an amino acid or a peptide substituted by or comprising an atom or a group of atoms capable of π-interactions with another same or different group in another peptide, thereby resulting in π-interactions or stacking. In some embodiments, the pi-pi interacting moiety is an aromatic group; thus the amino acid may be an aromatic amino acid or an amino acid modified by an aromatic group. Non-liming examples of aromatic amino acids include phenylalanine, tryptophan, tyrosine, histidine, 5-hydroxytryptophan ((5-HTP) also known as oxitriptan (INN)) or L-DOPA (L-3,4-dihydroxyphenylalanine).

In some embodiments, the aromatic amino acid is selected from phenylalanine, tryptophan, tyrosine and histidine. In some other embodiments, the aromatic amino acid is phenylalanine. In some embodiments, the pi-pi interacting moiety comprises phenylalanine. In some embodiments, the moiety is FF.

Any of the amino acids making up a peptide utilized according to the invention may be selected from naturally occurring and synthetic amino acids (non-natural amino acids), as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” are compounds that have the same fundamental chemical structure as naturally occurring amino acids, i.e., an alpha carbon that is bound to a hydrogen, a carboxyl group, an amino group, or an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” are chemical compounds that have structures that are different from the general chemical structure of an amino acid, but that function in a manner similar to the naturally occurring amino acids.

Amino acids may be referred to herein by either their commonly known three letter symbols or by the one-letter symbols recommended by the IUPAC-IUB Biochemical Nomenclature Commission.

In some embodiments, the amino acid is selected amongst natural amino acids, non-natural amino acids and any combination thereof. In some embodiments, the amino acid is at least one natural amino acid. In some other embodiments, the amino acid is at least one of alanine, asparagine, aspartate, cysteine, glutamate, glutamine, glycine, proline, serine, tyrosine, arginine, histidine, isoleucine, leucine, lysine, methionine, phenylalanine, threonine, tryptophan, and valine. In some embodiments, the amino acid is selected amongst D-amino acids. In some other embodiments, amino acid is selected amongst L-amino acids. In some embodiments, the amino acid is not a D-amino acid.

In some embodiments, where a hydrophilic amino acid is used, it may be a charged amino acid (positively charged or negatively charged).

In some embodiments, the hydrophilic amino acid is a negatively charged amino acid. In some embodiments, the hydrophilic amino acid is selected from aspartic acid and glutamic acid.

In some embodiments, the hydrophilic amino acid is a positively charged amino acid. In some embodiments, the hydrophilic amino acid is an amine substituted amino acid. In some embodiments, the hydrophilic amino acid is selected from lysine, arginine and histidine. In some other embodiments, the hydrophilic amino acid is lysine.

In some embodiments, the amino acid is an aromatic amino acid. Such amino acids may be selected from phenylalanine, tryptophan, tyrosine, histidine, 5-hydroxytryptophan ((5-HTP) also known as oxitriptan (INN)) and L-DOPA (L-3,4-dihydroxyphenylalanine). In some embodiments, the aromatic amino acid is selected from phenylalanine, tryptophan, tyrosine and histidine. In other embodiments, the aromatic amino acid is phenylalanine or derivatives thereof.

In a peptide utilized according to the invention, the N-terminus and/or the C-terminus may be each capped or substituted with an end capping group. The capping group may be any functional group or protecting group. In some embodiments, the peptide is of the amino acid sequence SEQ ID NO:1 or amino acid sequence SEQ ID NO:2, wherein at least one of the terminal amino acids is substituted to provide a capped peptide of the formula R₁-X₁₍ₙ₎-X₂₍ₘ₎-X₃₍ₚ₎-X_{4(q)}-R₂ (SEQ ID NO: 3), or the formula R₁-X₁₍ₙ₎-X₂₍ₘ₎-X₃₍ₚ₎-X_{4(q)}-R₂ (SEQ ID NO:4), or the formula R₁-X₀-X₂₍ₘ₎-X₃₍ₚ₎-X_{4(q)} (SEQ ID NO: 5), or the formula X₀-X₂₍ₘ₎-X₃₍ₚ₎-X_{4(q)}-R₂ (SEQ ID NO: 6) or the formula R₁-X₁₍ₙ₎-X₂₍ₘ₎-X₃₍ₚ₎-X_{4(q)} (SEQ ID NO:7), or the formula X₁₍ₙ₎-X₂₍ₘ₎-X₃₍ₚ₎-X_{4(q)}-R₂ (SEQ ID NO: 8), wherein R₁ is an N-capping group, and R₂ is a C-capping group. In any of the above sequences, X₀, X₁, X₂, X₃ and X₄ is an amino acid, such that optionally each one of X₁, X₂ and X₄ is independently selected from amino acids having a pi-pi interacting moiety and X₃ is a hydrophilic amino acid and wherein at each integer n, m, p and q is independently selected from 1 to 3; or each one of X₁, X₂ and X₄ is independently selected from amino acids having a pi-pi interacting moiety and X₃ is a hydrophilic amino acid and wherein at each integer n, m, p and q is 1 and wherein X₀ may be absent.

The capping groups may be selected in a non-limiting fashion from tert-butyloxycarbonyl (Boc), 9-fluorenylmethyloxycarbonyl (Fmoc), benzyloxycarbonyl (Cbz), p-methoxybenzyl carbonyl (Moz or MeOZ), trifluoroacetyl, benzyl, tert-butoxycarbonyl, p-methoxybenzyl (PMB), 3,4-dimethoxybenzyl (DMPM), p-methoxyphenyl (PMP), amide, acyl, acetyl (Ac), tosyl (Ts), benzoyl (Bz), Troc (trichloroethyl chloroformate), benzyl (Bn), carbamate, methoxy and methyl esters, benzyl esters, tert-butyl esters, silyl esters, orthoesters, oxazoline, hydroxyl, thiol, halide, alkyl, aryl, alkoxy, aryloxy, 2-naphthylmethyl ether (Nap) and amide. In some embodiments, the N-capping group is benzyloxycarbonyl (Cbz), p-methoxybenzyl carbonyl (Moz or MeOZ), tert-butyloxycarbonyl (Boc), 9-fluorenylmethyloxycarbonyl (Fmoc), acetyl (Ac), benzoyl (Bz), benzyl (Bn), carbamate, p-methoxybenzyl (PMB), 3,4-dimethoxybenzyl (DMPM), p-methoxyphenyl (PMP), tosyl (Ts), Troc (trichloroethyl chloroformate) or trifluoroacetyl, benzyl or 2-Naphthylmethyl ether (Nap). In some embodiments, the N-capping group is acetyl (Ac), benzyloxycarbonyl (“Cbz”) or 9-fluorenylmethyloxycarbonyl (Fmoc).

In some embodiments, the C-capping group is a methyl ester, a benzyl ester, a tert-butyl ester, a silyl ester, an orthoester or oxazoline.

In some embodiments, the C-terminus capping group is an amide, i.e., the carboxylic acid group of the C-terminus may be modified to an amide group.

In some embodiments, the pi-pi interacting moiety is a capping group, e.g., an aromatic group. In some embodiments, the aromatic capping group is 9-fluorenylmethyloxycarbonyl (Fmoc) or carboxybenzyl (Cbz). In some embodiments, the aromatic capping group is Cbz.

Without wishing to be bound by theory, it is suggested that aromatic capping groups may contribute to the overall interactions resulting in self-assembly of the peptides into nanostructures. These interactions may involve pi-pi interaction as well as hydrogen interactions. While the nature of interactions is not limiting, it is nevertheless believed to be highly stabilizing, amino acid selections as disclosed herein have been found to yield stable nanostructures that nonetheless undergo selective cleavage by the protease. As disclosed herein, peptides in accordance with the invention may be structured from 3 or more amino acids. In some embodiments, the peptides comprise three amino acids, e.g., tripeptides, wherein two of the amino acids may be aromatic amino acids and one amino acid is a hydrophilic amino acid. Similarly, the peptides may comprise 4 amino acids, i.e., tetra peptides, optionally comprising three aromatic amino acids and a single hydrophilic amino acid.

In some embodiments, the peptide is constructed of phenylalanine and lysine.

In some embodiments, the peptide comprises the motif phenylalanine-lysine-phenylalanine (F—K—F).

In some embodiments, the peptide is a tripeptide. In some embodiments, the peptide is a tripeptide denoted herein TPS5. In some other embodiments, the peptide is Fmoc-FKF-NH₂ (SEQ ID NO: 15).

In some embodiments, the peptide comprises the motif phenylalanine-phenylalanine-lysine-phenylalanine (F—F-K-F).

In some embodiments, the peptide has the amino acid sequence NH₂-FFKF-OH (SEQ ID NO:9), Ac-FFKF-OH (Ac-FFKF, SEQ ID NO:10), Fmoc-FFKF-OH (Fmoc-SEQ ID NO:11), Cbz-FFKF-OH (Cbz-FFKF, SEQ ID NO:12), Fmoc-FFKF-NH₂ (SEQ ID NO:13) or Cbz-FFKF-NH₂ (SEQ ID NO:14).

In some embodiments, the at least one peptide is a peptide of SEQ ID NO:9 (denoted TPS1), a peptide of SEQ ID NO:10 (denoted TPS2), a peptide of SEQ ID NO:11 (denoted TPS3), a peptide of SEQ ID NO:12 (denoted TPS4), a peptide of SEQ ID NO:13 (denoted TPS9) or a peptide of SEQ ID NO:14 (denoted TPS8). In some embodiments, the peptide is not TPS3.

As described herein, the peptides self-assemble into ordered arrangements that may be in a form such as nanoparticle, nanotube, nanofiber, nanowire or nanorod. In some embodiments, the nanostructure is a nanofiber. In some embodiments, the nanostructure, e.g., nanofiber, comprises at least one peptide of the formula NH₂-FFKF-OH (SEQ ID NO:9), Ac-FFKF-OH (SEQ ID NO:10), Fmoc-FFKF-OH (SEQ ID NO:11), Cbz-FFKF-OH (SEQ ID NO:12), Fmoc-FFKF-NH₂ (SEQ ID NO:13) or Cbz-FFKF-NH₂ (SEQ ID NO:14). In some embodiments, the nanostructure is not of the peptide or does not comprise the peptide TPS3.

In some embodiments, the nanostructure, e.g., nanofiber, comprises the peptide of SEQ ID NO:12 (denoted TPS4).

Nanostructures of the invention are recognized and cleaved by a protease. Generally speaking, the protease can be classified into seven groups: serine proteases using a serine alcohol; cysteine proteases using a cysteine thiol; threonine proteases using a threonine secondary alcohol; aspartic proteases using an aspartate carboxylic acid; glutamic proteases using a glutamate carboxylic acid; metalloproteases using a metal such as zinc and asparagine proteases using an asparagine. In some embodiments, the protease is a serine protease. In some embodiments, the protease is a cysteine protease. In some embodiments, the protease is an aspartic protease. In some embodiments, the protease is a metalloprotease.

In some embodiments, the protease is cathepsin.

As shown in FIG. 1 and Table 1, the peptide is recognized and cleaved by a protease such as cathepsin, present either within a cell or secreted from cells. Cathepsins are a diverse family of proteases found in all animals as well as other organisms and include different members, which are distinguished by their structure, catalytic mechanism and which proteins they cleave. Cathepsins include serine cathepsins (include Cathepsin A and Cathepsin G), aspartyl cathepsins (include Cathepsin D and Cathepsin E) and cysteine cathepsins. Cysteine cathepsins, are a family of proteases that share a conserved active site formed by at least one of cysteine, aspartic acid and histidine and include cathepsins B, cathepsin C, cathepsins F, cathepsins H, cathepsins K, cathepsins L1, cathepsins L2, cathepsins O, cathepsins S, cathepsins W, cathepsins Z (X). Out of the eleven cysteine cathepsins, the three major cathepsins, B, L and S are highly expressed by the tumor stroma cells mainly by tumor-associated macrophages (TAMs) of the microenvironment. Cathepsins are known to regulate several biological processes including, inflammation, antigen presentation, epidermal homeostasis, angiogenesis, extracellular matrix (ECM) turnover and more. In addition, cathepsins are overexpressed in various pathologies such as arthritis, osteoporosis, atherosclerosis, cancer and immune-related diseases. The cysteine cathepsin proteases play critical roles in several cancer processes including angiogenesis, degradation of vascular basement membrane, and activation of angiogenic growth factors. In addition, they degrade ECM components such as collagen IV, fibronectin and laminin. Degradation of such matrices during tumor invasion play a key role in metastasis by promoting migration of malignant cells out of primary tumors. Studies of tumor tissue and cell lines have shown changes in expression, activity and distribution of the cysteine cathepsins in numerous human cancers. Furthermore, the levels, activity and localization of the cathepsins have been shown to be of diagnostic and prognostic values. Several cysteine cathepsins have been found to be highly expressed and highly active in several cancer tissues such as in melanoma, colorectal, glioma, breast, lung and others.

In some embodiments, the cathepsin is a cysteine cathepsin. In some embodiments, the cathepsin is cathepsin L. In some embodiments, the cathepsin is cathepsin B.

In some embodiments, the nanostructures are of peptides listed in Table 1.

The interaction between the peptide and cathepsin involves a peptide bond cleavage, as schematically shown in FIG. 2J. Non-limiting examples of amino acid sequences recognized by cathepsin include FKF, FRF, YKF, YRF and FRH or any of the other sequences provided herein. In some embodiments, cathepsin cleaves a peptide bond between the residue being N-termini to the peptide bond and the residue being C-termini to the peptide bond. In some embodiments, cathepsin cleaves a peptide bond between the residue being C-termini to the peptide bond and the residue being N-termini to the peptide bond.

The nanostructures of the invention, being in some embodiments in the form of nanofibers, are capable of interacting (attaching, associating) with an agent such as a therapeutic agent or an imaging agent. The agent may be contained within the nanostructure or on its surface and released from the nanostructure, e.g., by cleaving the nanostructure. The cleavage may be in vivo by an enzyme, and thus the agent may be released in regions of increased enzyme expression or increased enzyme presence, providing in-site or local treatment of pathologies associated with the increased enzyme expression. The enzyme described herein may be a protease (also called a peptidase or proteinase) that hydrolyze a peptide bond. Thus, the nanostructures, e.g., nanofibers described herein, may be used for a modulated release of at least one agent at a predetermined location (tissue) and specifically to regions in the body associated with increased cathepsin presence and/or increased cathepsin activity.

In accordance with some aspects of the present disclosure, the nanostructure described herein is engineered or adapted for use as a vehicle or carrier of at least one agent. In other words, it may be used as a biocompatible drug delivery system for a variety of agents that can be directly delivered to loci of high enzyme, e.g., cathepsin, expression and/or presence.

In some embodiments, the drug delivery system is in the form of a nanofiber comprising a plurality of peptides, as detailed herein, and at least one agent, such that the nanofiber and/or the peptides making the nanofiber are associated with the agent. The association may be chemical or physical association or a bonding interaction, such as ionic bonding, covalent bonding, coordination bonding, complexation, hydrogen bonding, van der Waals bonding, hydrophobicity-hydrophilicity interactions, etc. In some embodiments, the association between the nanofiber and the agent is non-covalent.

In some embodiments, the agent adheres (coats or attaches) to the outer surface of the nanostructure, e.g., nanofiber. In some embodiments, the agent is encapsulated (embedded or entrapped) within the structure or inside a void in the structure. In some embodiments, some of the agent molecules are embedded or entrapped in the nanostructure while others may be associated with its surface. In some embodiments, the peptides coat the agent. In some other embodiments, the agent forms a fiber or a spherical structure that is coated by the peptides.

The association is further illustrated in the schematic representation of FIG. 3.

The agent may be a pharmaceutically active agent, an imaging moiety, a protein, a polypeptide, a nucleic acid molecule or an antibody. In accordance with some embodiments, the agent is an imaging moiety, an antibody or an aptamer and the nanostructure may be used for diagnosis of a variety of pathologies that are associated with increased cathepsin expression, namely conditions in which cathepsins are over-expressed, or conditions in which cathepsins play a role.

In accordance with some embodiments, the agent is a pharmaceutically active agent. The pharmaceutically active agent may be selected from small molecules, aptamer and antibodies. In accordance with such embodiments, the nanostructure may be used for treating, preventing, inhibiting, reducing, eliminating, protecting or delaying onset of a variety of pathologies that are associated with increased cathepsin expression, namely conditions in which cathepsin is over-expressed, or conditions in which cathepsin plays a role. Such a role can be directly related to pathological conditions or can be indirectly related to such a particular condition.

In some embodiments, the agent is a pharmaceutically active agent (drug). In some other embodiments, the drug is used for treating, preventing, inhibiting, reducing, eliminating, protecting or delaying the onset of a pathology associated with increased cathepsin expression.

Thus, the invention further provides a nanostructure, as described herein, associated with at least one agent for use in delivery or release of said at least one agent to regions of high cathepsin presence and/or activity. The invention further provides a drug delivery system comprising a nanostructure as described herein and at least one drug associated with said nanofiber for release of at least one drug in a region of high cathepsin presence and/or activity.

In accordance with some other aspects, there is provided a method of treating, preventing, inhibiting, reducing, eliminating, protecting or delaying the onset of a pathology associated with increased cathepsin expression or increased cathepsin presence in a subject in need thereof, the method comprises administering to the subject a composition comprising a nanostructure associated with at least one drug, as described herein.

Thus, the present disclosure also provides in accordance with some aspects, a composition comprising nanostructures, e.g., nanofibers, as described herein, and at least one agent. The composition according to some embodiments is a pharmaceutical composition. In some embodiments, the composition is a pharmaceutical composition for use in treating, preventing, inhibiting, reducing, eliminating, protecting or delaying the onset of a pathology associated with increased cathepsin expression or increased cathepsin presence in need thereof. In some embodiments, the pathologies that are associated with increased cathepsin are proliferative disorders. In some embodiments, the pathologies that are associated with increased cathepsin are inflammatory disorders. In some embodiments, the pathologies that are associated with increased cathepsin is arteriosclerosis or Vulnerable plaques.

A proliferative disorder is a disorder displaying cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper-proliferative disease” or “hyper-proliferative disorder.” It should be noted that the term “proliferative disorder”, “cancer”, “tumor” and “malignancy” all relate equivalently to a hyperplasia of a tissue or organ. Non-limiting examples of cancers include blastoma, carcinoma, lymphoma, leukemia, sarcoma, mesothelioma, glioma, germinoma, choriocarcinoma, melanoma, glioblastoma, lymphoid malignancies, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, esophageal cancer as well as head and neck cancer.

An inflammatory disorder is a disorder encompassing any immune response. The inflammatory disorder may be an infectious or a non-infectious disorder. Non-infectious inflammatory disorders are any disorder which the activation of macrophages or activated macrophages play a role such as auto-immune disorders and inflammatory disorders which are not infection related, i.e. non-pathogenic, caused by other than an infectious agent (e.g. auto-antigen, hypersensitivity, wound). Not limiting examples include inflammatory diseases of the gastrointestinal tract such as Crohn's disease, inflammatory bowel disease, gastritis, colitis, ulcerative colitis, colon irritable, gastric ulcer and duodenal ulcer, inflammatory diseases of the skin such as psoriasis, inflammatory diseases of the respiratory system such as asthma, allergic rhinitis or chronic obstructive pulmonary disease (COPD), pulmonary fibrosis, sarcoidosis, inflammatory diseases of the musculoskeletal system such as rheumatoid arthritis, osteomyelitis, osteoporosis, or neuritis, systemic sclerosis, inflammatory diseases of the kidneys such as glomerulonephritis, renal ischemia, or renal inflammation; inflammatory diseases of the nervous system such as multiple sclerosis, Alzheimer's disease and HIV-1-associated dementia; autoimmune diseases such as diabetes, type 1 and 2 diabetes mellitus and graft versus host reaction; infectious disease such as nephritis, sepsis, septic shock, endotoxic shock, adult respiratory distress syndrome; inflammatory conditions of the cardiovascular system, such as myocardial infarction, myocarditis, atherosclerosis, hypertensive cardiomyopathy, atheroma, intimal hyperplasia or restenosis or autoimmune disorders such as Multiple Sclerosis (MS), inflammatory arthritis, rheumatoid arthritis (RA).

In some embodiments, the pathologies that are associated with increased cathepsin are any one of the above-mentioned cancers. In some embodiments, the pathologies that are associated with increased cathepsin are pathologies associated with angiogenesis, degradation of vascular basement membrane, and activation of angiogenic growth factors, macrophage-targeted treatment.

In some embodiments, the nanostructure comprises at least one peptide denoted herein TPS4 and an anticancer drug. In some embodiments, the nanostructure comprises at least one peptide having an amino acid sequence Cbz-FFKF-OH and an anticancer drug. In some embodiments, the nanostructure comprises at least one peptide having the sequence SEQ ID NO:12 and an anticancer drug. Non-limiting examples of anticancer drugs include chemotherapeutic drugs, hormonal therapy and drugs used in immunotherapy. In some embodiments, the drug delivery system described herein comprises nanostructures comprising at least one peptide and a chemotherapeutic drug. In some embodiments, the nanostructure comprises at least one peptide denoted herein TPS4 and a chemotherapeutic drug.

In some embodiments, the pathologies that are associated with increased cathepsin are skin disorders. In further embodiments, the pathologies that are associated with increased cathepsin are cardiovascular diseases. In some embodiments, the pathologies that are associated with increased cathepsin are selected from atherosclerosis, osteoarthritis, arthritis, Alzheimer's disease and psoriasis. In some further embodiments, the pathologies that are associated with increased cathepsin are selected amongst parasite infections.

In accordance with some embodiments, the agent is an imaging agent, an antibody or an aptamer and the nanostructure may be used for diagnosis of a variety of pathologies that are associated with increased cathepsin expression, namely conditions in which cathepsins are over-expressed, or conditions in which cathepsins play a role. Thus, the present disclosure also provides a method of diagnosing a pathology associated with increased cathepsin presence/activity in a subject in need thereof, the method comprising administering to the subject a composition comprising a nanostructure as described herein associated with at least one diagnostic agent, and identifying regions in the subject's body where the agent has been localized.

The method of diagnosis may also be used for determining the effect of an existing treatment regimen, for determining the severity of a disease or condition, etc.

The term “treatment or prevention” concerns improvement of at least one undesired manifestation of the disease such as increase in disease free periods, decrease in acute disease periods (in time and severely), decrease in severity of the disease, improvement in life quality, decreased mortality, decrease in the rate of disease progression as well as prophylactic treatment before disease occurs. More specifically, the term concerns a complete range of therapeutically positive effects of administrating to a subject including inhibition, reduction, alleviation and relief from a disorder or any related condition and illness, symptoms or undesired side effects or related disorders. These include ameliorating existing symptoms, preventing additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

By “patient” or “subject in need” it is meant any mammal, e.g., human subject, who may be affected by the above-mentioned conditions, and to whom the treatment and diagnosis methods herein described is desired, including human, bovine, equine, canine, murine and feline subjects. Preferably said patient is a human. Administering of the therapeutic agent to the patient includes both self-administration and administration to the patient by another person. It should be noted that treatment according to the invention, would ameliorate or decrease in acute disease periods (in time and severely), decrease in severity of the disease, or even prevent. It should be appreciated that a subject as defined herein is also applicable for any of the methods of the invention described in further aspects of the invention.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

According to some other aspects, the present disclosure provides a kit comprising at least one nanostructure according to the invention and instructions of use. The nanostructure being in a form suitable for administration to a human subject. The form may be selection of suspension, powder ready for dissolution, an injectable solution and others. The kit may further comprise a solution for solubilizing the nanostructure (e.g., in the form of a solid powder). The nanostructure may be associated, as disclosed herein, with at least one drug or imaging agent. According to such embodiments, the kit of the invention may be for use in a method for treating, preventing, ameliorating or delaying the onset of a disease associated with increased expression or presence of cathepsin and associated pathologies in a subject in need thereof; or for use in a method of diagnosis.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C are representative HPLC chromatograms showing cleavage of a tetra-peptide substrate (TPS) by cathepsins, each TPS was treated with cathepsin B, cathepsin L or a vehicle at 37° C. and cleavage products were analyzed by liquid chromatography-MS (LC-MS). FIG. 1A—TPS3 absorbance at 215 nm, FIG. 1B—TPS3 after cathepsin B treatment, FIG. 1C—TPS3 after cathepsin L treatment.

FIG. 2A-K show structure of exemplary TPS peptides utilized in accordance with the invention. FIGS. 2A-2I show structures of TPSs based on the FFKF scaffold, each carrying a unique N-termini chemical group and a variation in the C-termini charge: TPS1 (FIG. 2A), TPS2 (FIG. 2B), TPS3 (FIG. 2C), TPS4 (FIG. 2D), TPS6 (FIG. 2E) and TPS7 (FIG. 2F), TPS9 (FIG. 2G), TPS8 (FIG. 2H) and TPS5 (FIG. 2I). FIG. 2J provides a schematic representation of protease binding pockets (S) and corresponding substrate amino acid (P), the cleavage site is the scissile bond between the P1 (N-termini to the scissile bond) and P1′ (C-termini to the scissile bond) residues. FIG. 2K shows exemplary synthesis of TPSs carried out using Fmoc based solid phase peptide synthesis. In the scheme: Fmoc is 9-fluorenylmethylcarbonyl; Boc is t-butyloxycarbonyl; Cbz is carboxybenzyl; TFA is trifluoroacetic acid.

FIG. 3 is a schematic representation of an exemplary nanostructure and an agent according with some embodiments.

FIGS. 4A-I are TEM micrographs showing self-assembly of TPSs triggered by solvent-switching between DMSO and water, final concentration of 5 mg/ml of TPS1 (FIG. 4A), TPS2 (FIG. 4B), TPS3 (FIG. 4C), TPS4 (FIG. 4D), TPS5 (FIG. 4E), TPS6 (FIG. 4H) and TPS7 (FIG. 4I) and 1 mg/ml of TPS8 (FIG. 4F) and TPS9 (FIG. 4G) were analyzed. TEM analysis revealed self-assembled nanofibers with different morphologies for all TPSs, excluding TPS6 and TPS7 (FIGS. 4H and 4I, respectively).

FIGS. 5A-C provide a kinetic profile of TPS4 self-assembly at different concentrations, evaluated by turbidity measurements. after solvent switching from DMSO to water, absorbance at 350 nm was measured every two minutes during the first 5 hours, monitoring the self-assembly at 37° C. A significant increase in turbidity was observed in all concentrations tested (up to 2.8±0.6 folds), demonstrating continuous assembly of the nanofibers.

FIGS. 6A-E show TPS4 nanofibers degradation by cathepsin B, FIGS. 6A-C are graphs showing TPS4 assembly behavior, following assembly of TPS4 at the indicated peptide concentrations, and treatment with cathepsin B or a vehicle. The degradation of the assembled TPS4 was evaluated by turbidity measurements monitoring the absorbance at 350 nm, the addition of cathepsin B led to decreased turbidity due to enzyme-mediated degradation of the nanofibers. FIG. 6D is a bar representation showing quantification of absorbance at 350 nm indicating significant reduction in turbidity in cathepsin B treated samples the results described with standard deviation, ** p<0.01, *** p<0.001. FIG. 6E provides TEM images of TPS4 nanofibers degraded by cathepsin B, the nanofibers were analyzed at the indicated concentrations by TEM before and after cathepsin B treatment. A substantial reduction in the number of detected nanofibers was found in the treated samples (lower panel). Scale bar is 5 μM.

FIG. 7 is a bar representation of TPS4 nanofibers cytotoxicity. MDA-MB 231 cells were cultured in 96 well plate one day prior to treatment, triplicate samples of cells were incubated with 0.5 mg/ml TPS4 nanofibers in growth medium or with 0.1% DMSO vehicle control, after 24 or 48 hours treatment, cell survival was determined by standard methylene blue assay, the graphs shows that TPS4 nanofibers show no growth inhibition of MDA-MB 231 cells.

FIGS. 8A-C show TPS4 nanofibers as a targeted drug-release carrier. The assembly of premixed solution of Doxorubicin (Dox) and TPS4 in DMSO was triggered by solvent change and allowed for overnight self-assembly in the dark. FIG. 8A provides a bright field and FIG. 8B fluorescent microscopy (excitation 535±50 nm, emission 610±75 nm) images of the nanofibers. FIG. 8C is a graph representation showing the effect of cathepsin B or a vehicle added to samples from FIGS. 8A and 8B and released drug was collected for 4 hours by dialysis, the amount of released Dox was extrapolated from a fluorescent Dox calibration curve, results described with standard deviation.

FIGS. 9A-G show an improved drug release profile. Dox was precipitated by ammonium sulfate to generate Dox particles (DPs), TPS4 was added and allowed to assemble overnight. FIG. 9A and FIG. 9B are bright field and fluorescent microscopy images, respectively, of DP-TPS4. FIG. 9C and FIG. 9D are SEM and TEM images, respectively, of DPs alone showing that DPs form unstructured aggregates as well as fibers (white arrow). FIG. 9E and FIG. 9F are SEM and TEM images, respectively, of DPs-TPS4 showing defined nanofibers. FIG. 9G is a graph representation showing the release profile of Dox from DP-TPS4 nanostructure in the presence and absence of cathepsin B. After 8 hours, 91.8±0.3% of the drug was released from DP-TPS4 following cathepsin B treatment. Results are described with standard error, ** p<0.01, *** p<0.001, **** p<0.0001.

FIG. 10 is a bar representation showing degradation of TPS4 nanofibers by tissue lysates. Mice 4T1 tumor lysates or vehicle were mixed with TPS4 assemblies and after overnight treatment, the amount of remaining nanofibers relative to vehicle was determined by absorbance measurement at 350 nm, less than 20% TPS4 nanofibers remained following lysate treatments in all concentrations tested and reached full degradation at the lowest concentration.

FIG. 11 is a graph representation showing degradation of TPS4 nanofibers by tissue lysates, following 0.5 mg/ml TPS4 assembly, lysates from 4T1 tumors or from mice muscles were added and the degradation of the assembled TPS4 was evaluated by turbidity measurements, monitoring the absorbance at 350 nm, addition of 4T1 tumor lysates led to dramatic decreased turbidity resulting from enzyme-mediated degradation of the nanofibers, results described with standard error, * p<0.05, ** p<0.01, *** p<0.001.

### DETAILED DESCRIPTION OF EMBODIMENTS

### Non-Limiting Examples

### Results and Discussion

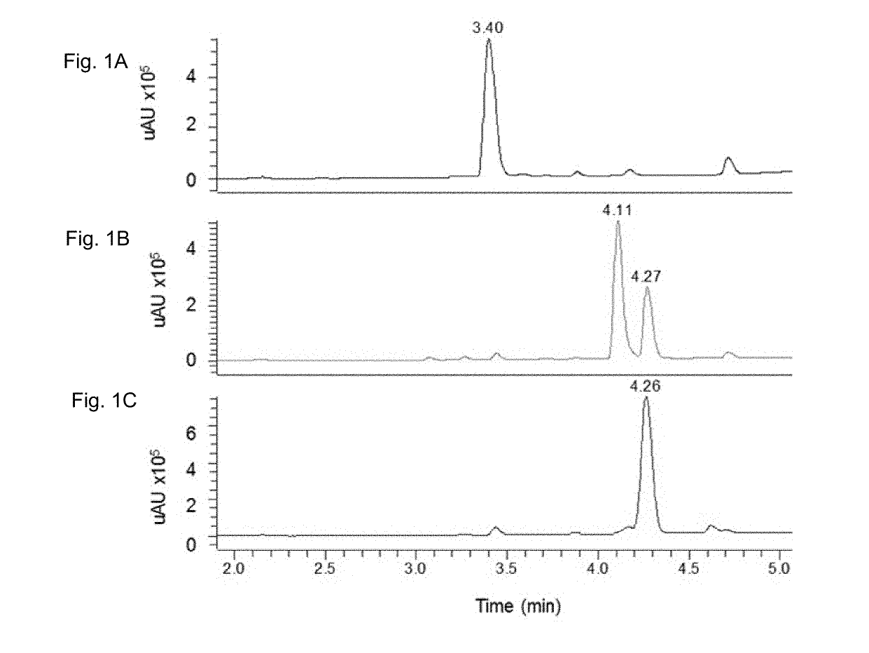

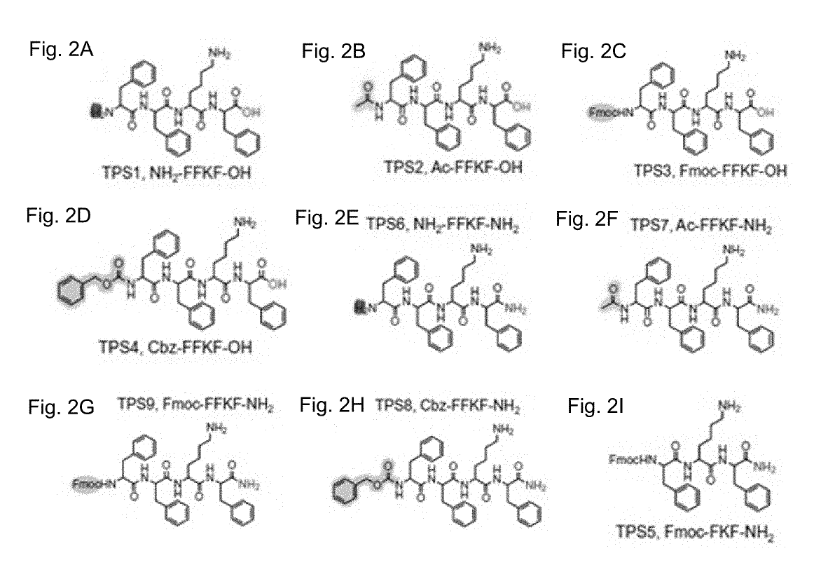

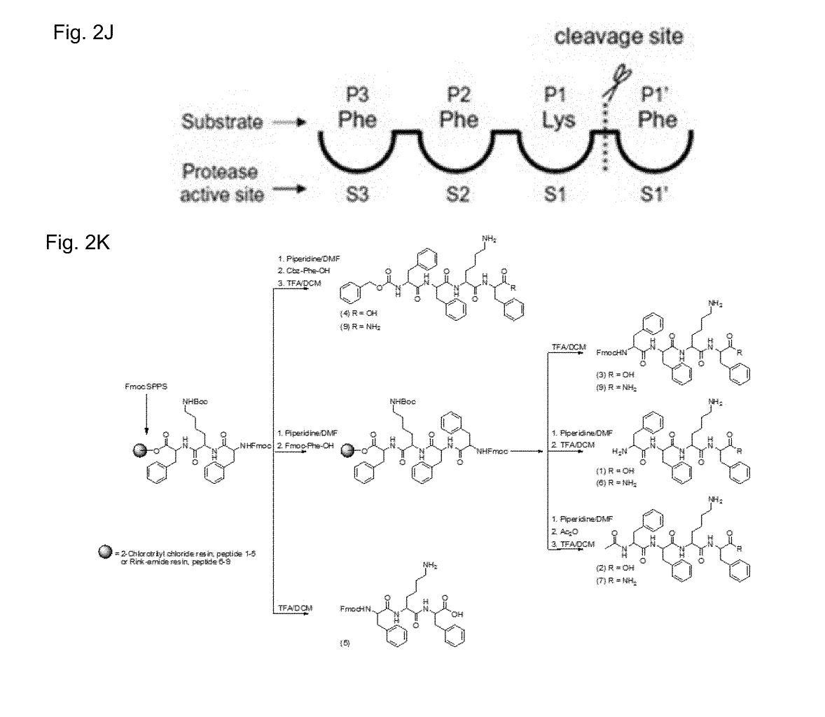

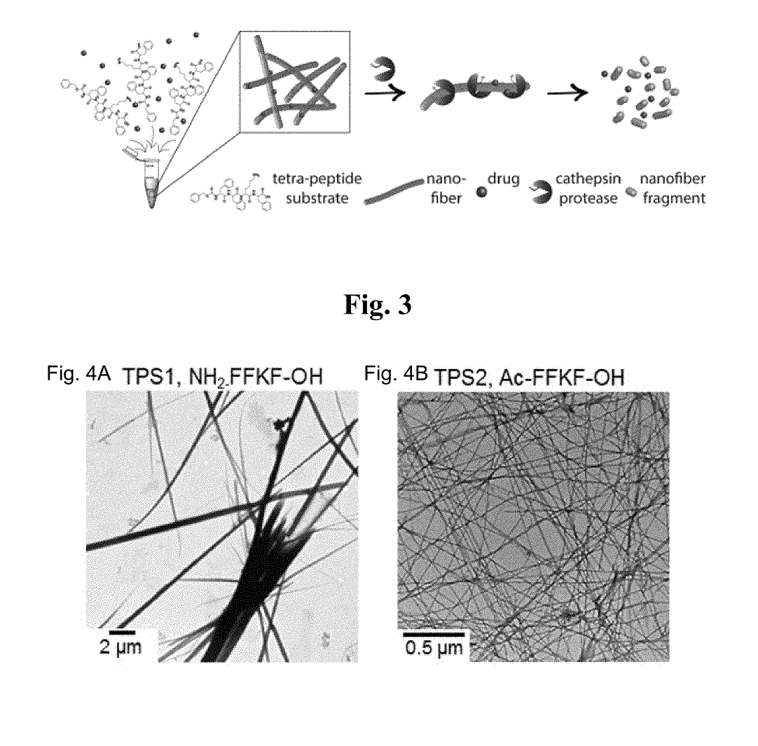

To target the delivery of therapeutics to cancerous tissues with elevated cathepsin activity, a series of TPSs based on the FFKF scaffold was designed. To optimize the TPSs self-assembly, the peptide designs contain unique N-termini chemical groups and charge variation at the C-termini (FIGS. 2A-2I). Aromatic chemical groups were introduced at the N- and C-termini to ensure that the addition of the lysine to the FF variant will not impair the self-assembly process. A tri-peptide fragment FFK was the expected cleavage product since the target site for cleavage by these cathepsins is postulated to be the amide bond after the P1 Lys, recognized by the protease S1 pocket (FIG. 2J). Following synthesis of these peptides (FIG. 2K), their ability to be recognized and cleaved by cathepsin proteases was studied. The peptides were incubated with either cathepsin B or cathepsin L and their cleavage products were analyzed using mass spectroscopy (MS). Results obtained from MS analysis revealed that all TPSs were recognized and cleaved by both enzymes. In addition to the expected tri-peptide cleavage product, di-peptides and/or single amino acids were detected (FIG. 1, Table 1), as accounted for by the known highly promiscuous nature of the cathepsin proteases. It is widely accepted that the sequence determines the substrate specificity to a protease, in some cases the inventors found correlations between the cleavage pattern and the peptide N-termini capping group. In cathepsin B for example, a charged amine group lead to removal of a single amino acid while bulky hydrophobic groups (Fmoc or Cbz) led to removal of two or three amino acids.

TABLE 1
Number of amino acids removed from each TPS after cathepsin
treatment according to the fragment analysis by LC-MS.

| TPS | Cat B | Cat L |
|---|---|---|
| TPS1 | 1 | 2 |
| NH₂-FFKF-OH | | |
| TPS2 | 2 | 2 |
| Ac-FFKF-OH | | |
| TPS3 | 2, 3 | 2 |
| Fmoc-FFKF-OH | | |
| TPS4 | 2 | 2 |
| Cbz-FFKF-OH | | |
| TPS5 | 1 | 1 |
| Fmoc-FKF-amide | | |
| TPS6 | 1 | 2 |
| NH₂-FFKF-amide | | |
| TPS7 | 1 | 2 |
| Ac-FFKF-amide | | |
| TPS8 | ND* | 2 |
| Cbz-FFKF-amide | | |
| TPS9 | 2, 3 | 1, 2 |
| Fmoc-FFKF-amide | | |

Cat; cathepsin,
ND; not determined,
*detected fragments identity could not be determined.

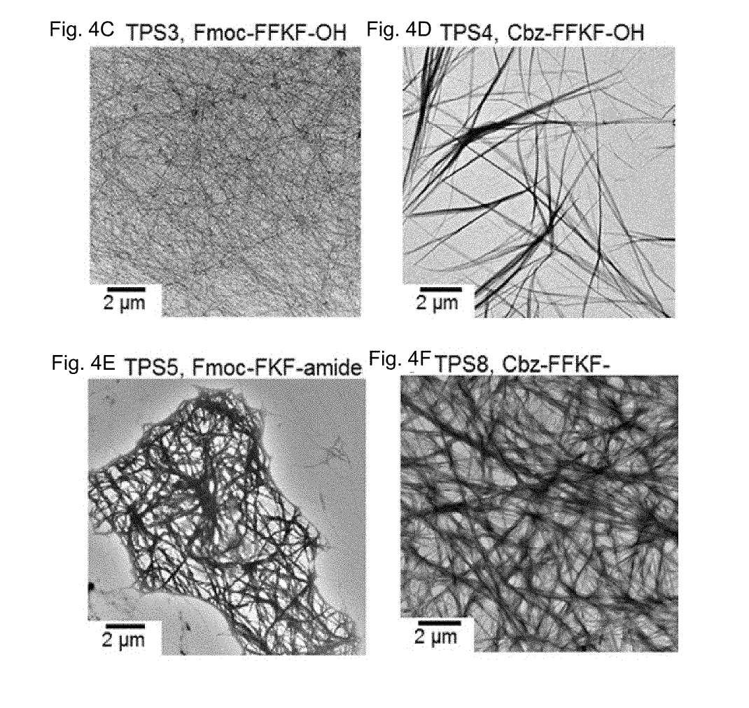

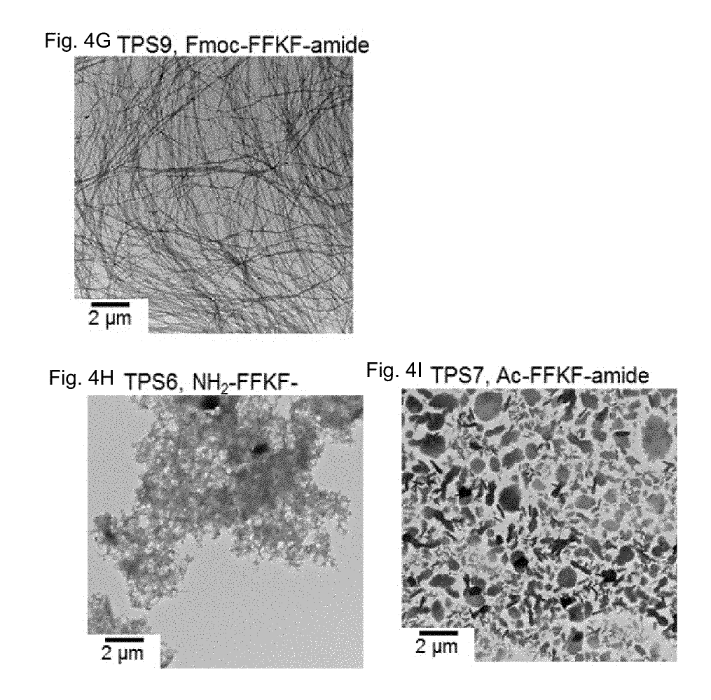

Next, the inventors investigated the ability of the different TPSs to self-assemble into ordered nanostructures using a solvent-mediated approach to trigger the assembly. Nanostructures formation was verified by transmission electron microscopy (TEM) and revealed that all TPSs self-assembled into ordered structures with morphology of elongated nanofibers network, excluding TPS6 and 7 (FIG. 4). In most cases the addition of a charged amino acid, Lys, to the fundamental FF structure did not prohibit structure formation, most likely because of the many aromatic rings within the TPSs. The inventors observed variance in the nanofibers diameters of the different TPSs (Table 2) that was attribute to the chemical modifications at the N- and C-termini Aromatic moieties (such as Fmoc and Cbz) and carbonyl/amide groups (FIG. 2) can contribute to the total π-π and hydrogen bond interactions, respectively. These additional interactions may enable stronger stacking forces yielding “well-packed” nanostructures with relatively smaller diameter. Interestingly, upon assembly initiation TPS4 stood out since it instantly formed fibers, while most other TPSs took over two hours to assemble, as was confirmed by microscopic examination (data not shown).

TABLE 2
Summary of TPSs diameters

| | TPS1 | TPS2 | TPS3 | TPS4 | TPS5 | TPS6 | TPS7 | TPS8 | TPS9 |
|---|---|---|---|---|---|---|---|---|---|
| Diameter | 310 ± 80 | 12 ± 1 | 14 ± 2 | 24 ± 6 | 66 ± 13 | — | — | 30 ± 6 | 13 ± 4 |
| (nm) | | | | | | | | | |

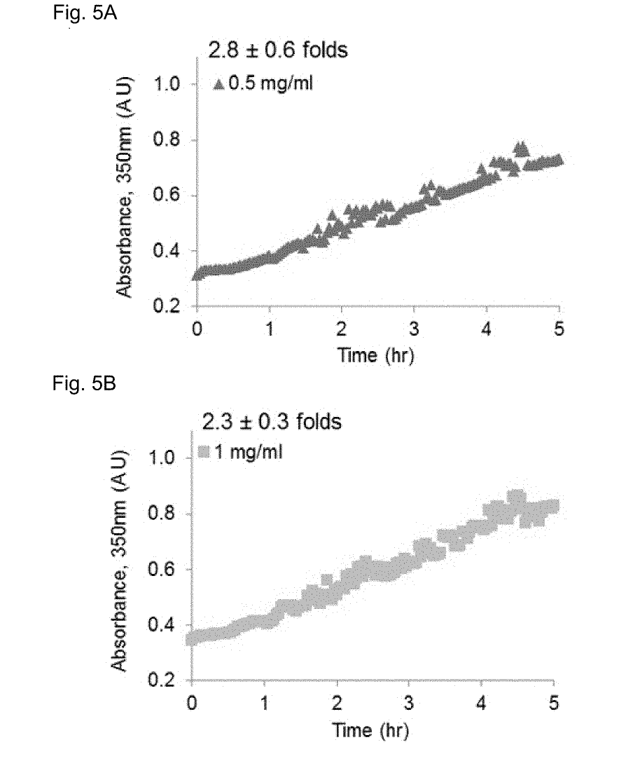

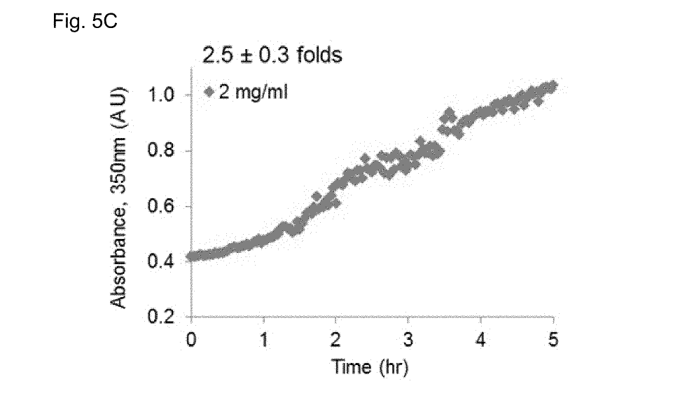

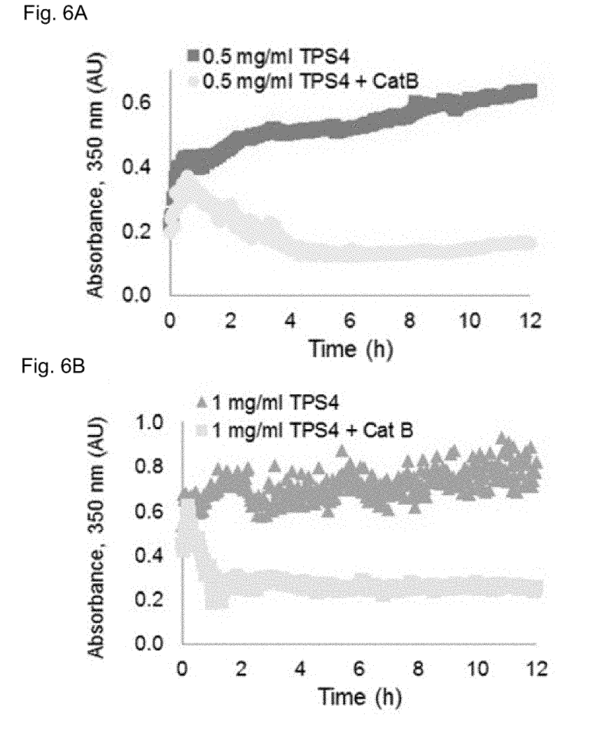

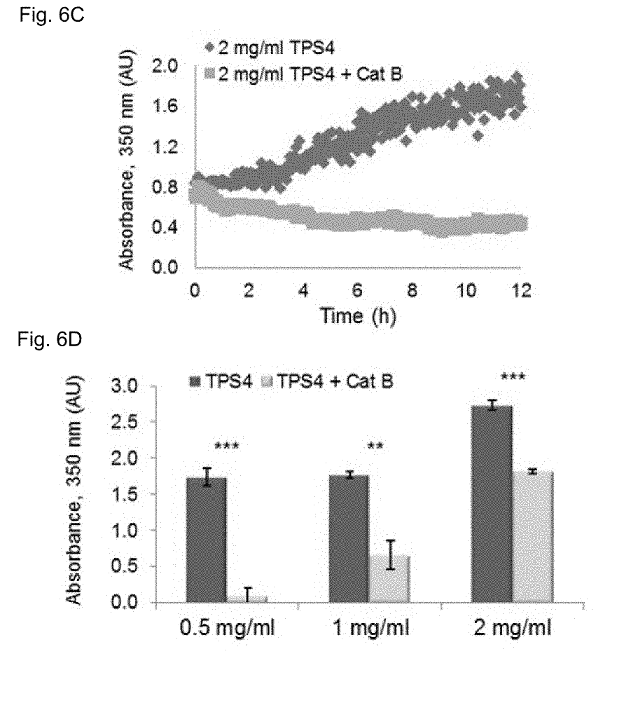

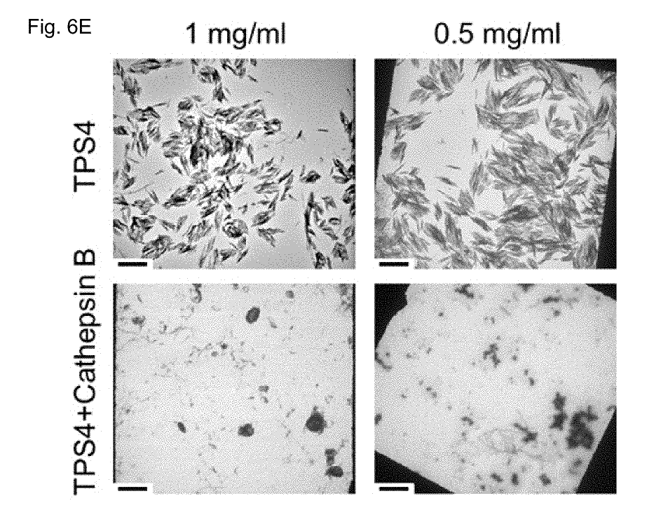

Considering its immediate self-assembly, the inventors continued with TPS4 (Cbz-FFKF-OH), as a self-assembling peptide-substrate model for cleavage by cathepsin B. TPS4 was found to assemble into ordered nanofibers, forming an opaque peptide-solution upon assembly initiation. To evaluate the assembly kinetics of TPS4 the inventors examined the turbidity changes of the self-assembled peptide at different peptide concentrations by monitoring the absorbance at 350 nm. Absorbance was already detected at the initial time point followed by a significant increase in turbidity during the first five hours (FIGS. 5A-C) indicating continuous assembly of the nanofibers over the course of the experiment. This increase in turbidity is in line with assembly of Boc-Phe-Phe, where nanofibers formation was associated with the increase in turbidity. Upon addition of cathepsin B to the assembled TPS4 a decrease in turbidity was obtained, due to the degradation of the nanofibers by the enzyme (FIG. 6A-C). To validate that the decrease in turbidity was a result of nanofiber degradation, samples of nanofibers with and without cathepsin B treatment were analyzed by TEM at the final time point. While very few nanofibers were found in the samples treated with cathepsin B, a substantial number of nanofibers were easily found in non-treated samples (FIG. 6E). This validated that cathepsin B can access TPS4 and degrade it even as assembled nanofibers.

The inventors further evaluated nanofiber degradation by cathepsin B at various TPS4 concentrations to examine the optimum peptide concentration for best assembly and degradation when the assembly process reached equilibrium. As expected, under the tested conditions a significant and robust degradation of TPS4 assemblies was obtained for all tested peptide concentrations. Most dramatic turbidity reduction was found at the lowest concentration tested, 0.5 mg/ml. At the higher TPS concentration, however, only partial degradation was observed (FIG. 6D). The inventors speculate that at higher peptide concentrations more assemblies are present in the solution that might physically limit the accessibility of the enzyme to the substrate. Another possibility is that the degradation products are quickly recovered and assembled to the remaining TPS structures that are present at a high concentration in the surrounding environment. Overall these results suggest that nanofibers degradation is concentration dependent and that TPS4 assemblies should be optimized to generate suitable drug delivery systems.

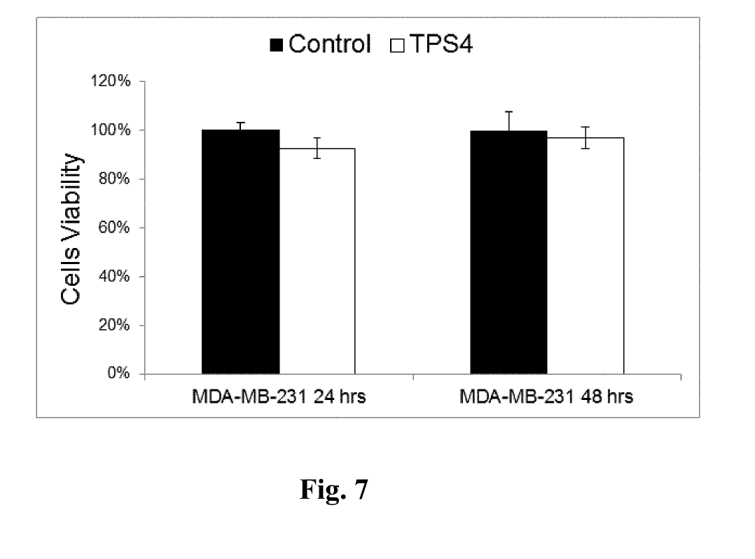

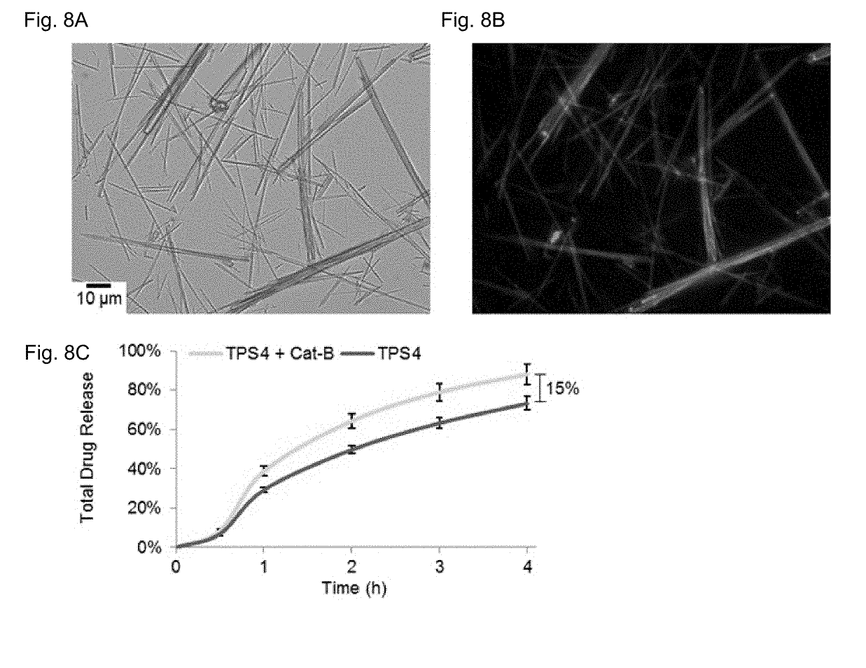

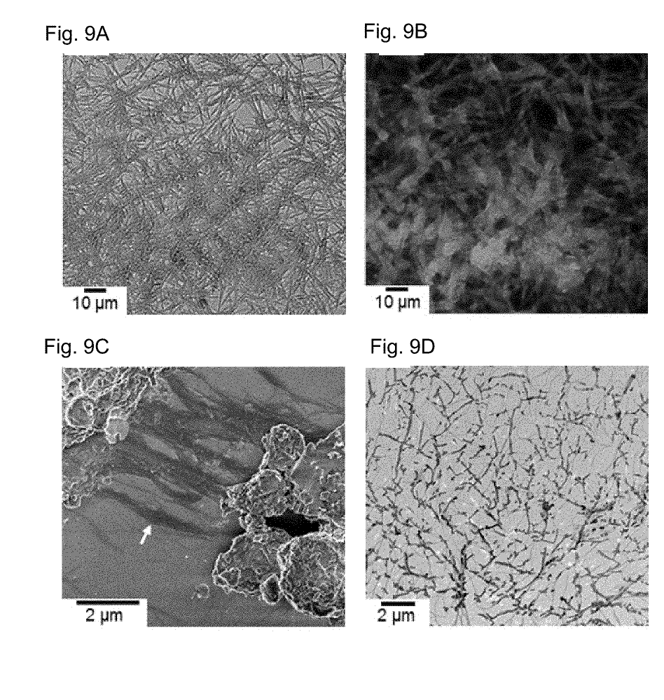

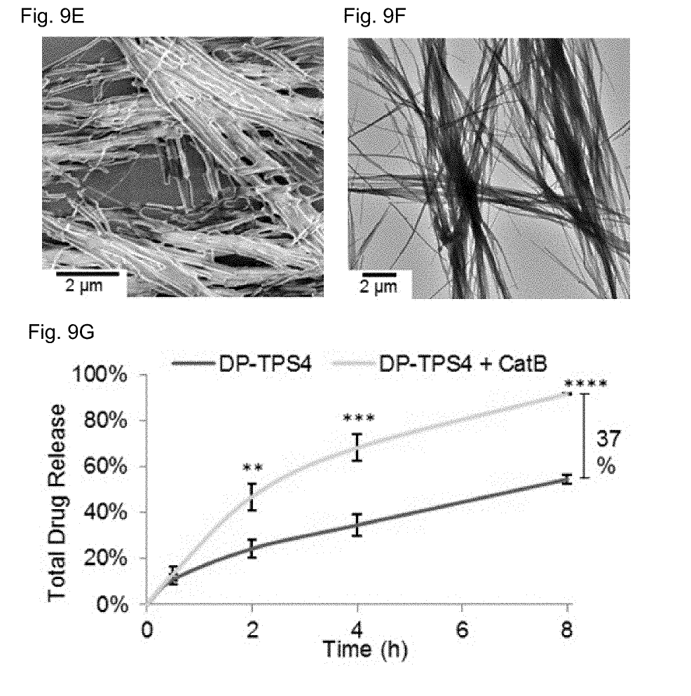

To further investigate the ability of TPS4 to serve as a cathepsin targeted drug delivery vehicle the inventors first evaluated growth inhibition by TPS4 and found no cytotoxicity of MDA-MB 231 cells by 0.5 mg/ml, (FIG. 7). Then the inventors assessed the ability of assemblies obtained from 0.5 mg/ml TPS4 to serve as a carrier for the anti-cancerous drug Doxorubicin (Dox). Dox was chosen as a model drug because of its intrinsic fluorescent properties which allow easy monitoring of the drug. Initially, Dox was encapsulated in nanofibers of TPS4 by solvent switching of the mixture from DMSO to water, generating fluorescent nanofibers as visualized by fluorescent microscopy (FIG. 8B). Exposure of the Dox containing nanofibers to cathepsin B resulted in drug release due to nanofibers degradation. Unfortunately, the inventors found only a 15% difference in the release profile when comparing the amount of drug released from the nanofibers with or without the enzyme (FIG. 8C). The inventors suspected that Dox was only coating the nanofibers by weak interactions and was naturally released thus leading to the small differences observed upon enzyme addition. To improve the release profile upon enzyme treatment, the inventors attempted to first generate small Dox particles and then coat them with peptide nanofibers. The Dox particles were inspired by the doxil liposome in which drug retention was achieved by base change of a weak-base-drug with sulfate ions in an intra-liposome aqueous phase. In that process, after accumulation in a liposome filled with ammonium sulfate, (doxorubicin)₂SO₄ (doxorubicin sulfate) precipitated. To investigate the hypothesis, the inventors first generated Dox particles (DPs) by precipitation with ammonium sulfate and then coated these particles with peptide nanofibers triggering the assembly of TPS4 by solvent exchange, Dox loading efficiency was found to be 48±5%. The new assembly, Dox particles-TPS4 (DP-TPS4), formed highly fluorescence structures, as observed by fluorescent microscopy (FIGS. 9A and 9B). Scanning electron microscopy (SEM) and TEM analysis of the DPs revealed differences in the structures formed with and without TPS4. DPs alone formed unstructured aggregates as well as fibers with 10-20 nm diameter (FIGS. 9C and 9D, marked by a white arrows). DPs-TPS4 formed defined nanofibers throughout, with fibers diameter ranging from 40 to 60 nm (FIGS. 9E and 9F). The inventors then turned to investigate the release profile of Dox from DP-TPS4 assemblies in the presence or absence of cathepsin B. As expected, in the presence of the enzyme a significant increase in Dox release was obtained that reached 91.8±0.3% after eight hours, as compared to the spontaneous, non-specific, drug release from DPs-TPS4 structures without enzyme treatment (55.0±0.2%) (FIG. 9G).

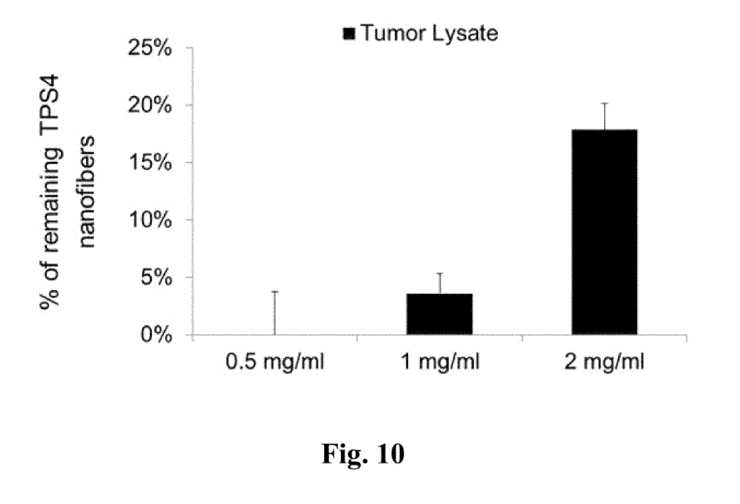

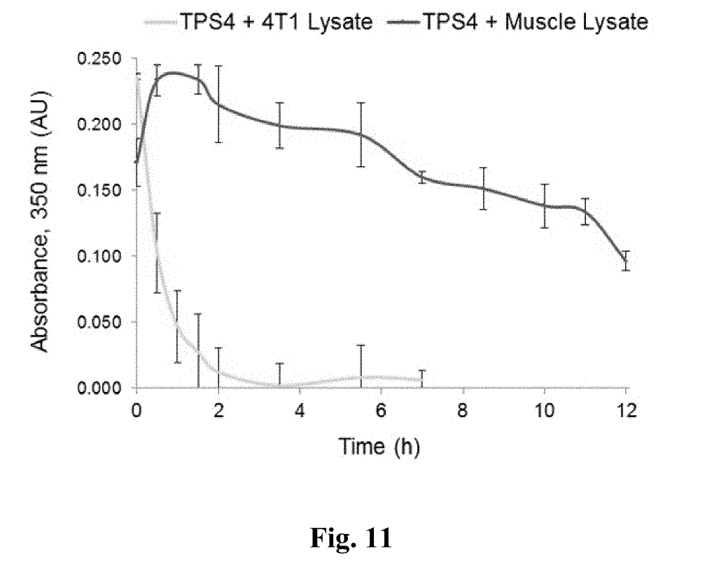

Further, the inventors analyzed the expression and activity levels of cathepsins in 4T1 murine breast cancer cells. Similar to other cancers, this cell line naturally expresses high levels of various cathepsins, especially cathepsin B. Therefore, the inventors evaluated the ability of 4T1 tumor lysates from tumors-bearing mice to degrade TPS4 nanofibers. The inventors found that the tumor lysates degraded TPS4 nanofibers in a concentration-dependent manner (FIG. 10). The inventors then evaluated TPS4 nanofiber degradation over time by tissue lysates, while 4T1 tumor lysates degraded the majority of TPS4 nanofibers within two hours, the lysates generated from mice muscles had limited effect in the first ten hours tested (FIG. 11). Taking these results together, the inventors foresee that tetra-peptide substrates that form nanostructures could serve as a promising platform for targeted drug delivery to cancers that exhibit highly elevated protease activities.

In conclusion, the inventors applied a substrate-based approach to generate a library of self-assembled tetra-peptides to serve as carriers for therapeutics to pathogenic tissues characterized by elevated protease activity. The inventors have demonstrated that in most cases elongation of the FF variant by two additional amino acids, including a charged lysine, did not impair the substrates self-assembling into ordered nanofibers. Furthermore, the inventors show the capability of the cathepsin proteases to process their substrate both in solution and within nanostructures. Generation of Dox particles-TPS4 led to an improved release profile of Dox from the nanostructures by cathepsin B activity. Finally, the inventors demonstrated that the intrinsic high cathepsins activity of tumor lysates can fully degrade TPS4 nanofibers. The findings described herein suggest a new platform for drug-delivery, targeted to pathologies with high cathepsins activity.

### Materials and Methods

### Chemical Synthesis

Unless otherwise noted, all resins and reagents were purchased from commercial suppliers and used without further purification. Tetra peptides were synthesis on solid phase using 2-Chlorotrityl chloride resin (peptide 1-5) or Rink-amide resin (peptide 6-9) with Fmoc based chemistry. Peptide elongation was performed with HOBT (1-hydroxybenzotriazole) and PyBOP (benzotriazole-1-yl-oxy) tris (pyrrolidino) phosphonium hexafluorophosphate) as coupling reagents and DIEA (diisopropylethylamine) Cleavage of peptide from the resin and Boc (t-butyloxycarbonyl) deprotection were performed using TFA (trifluoroacetic acid) in DCM (dichloro methane). Peptides were purified by C18 reverse phase HPLC in acetonitrile/water gradient supplemented with 0.1% trifluoroacetic acid (TFA) or by precipitation. Final peptides were characterized by a Liquid Chromatography Mass Spectrometer (LCMS—Thermo Scientific MSQ-Plus attached to an Accela UPLC system) to more than 95% purity. Final peptides were lyophilized and kept at −20° C. until use.

### Cleavage of Substrate Peptides by Cathepsins

Each peptide (1 nmole) was treated with 0.9 μmole cathepsin B or cathepsin L (generated by transformation of E. coli BL21(DE3)pLysS strain with the pET3a/procathepsin B or procathepsin L vectors) or acetate buffer vehicle (50 mM acetate, 5 mM MgCl₂, 2 mM DTT, adjusted to pH 5.5) for 3 hours at 37° C. Next, enzymes were precipitated by an hour incubation at −80° C. in 80% cold methanol followed by 10 minutes of 6000 g centrifugation at 4° C. Supernatant volume was reduced to about 15 μl under vacuum, adjusted to a final volume of 30 μl with methanol and analyzed by LCMS equipped with C18 reverse phase.

### Assembly Preparation

Lyophilized peptides were dissolved in DMSO to a concentration of 100 mg/ml and then diluted in DDW to the indicated final concentration.

### Transmission Electron Microscopy Analysis

Transmission electron microscopy (TEM) analysis was performed by applying 10 μl of samples to a 200 or 400-mesh copper grids covered by carbon-stabilized Formvar film (SPI, West Chester, Pa.). The samples were allowed to adsorb for 2 minutes before excess fluid was blotted off. Samples were negatively stained by depositing 10 μl of 2% uranyl acetate on the grid and allowing it to adsorb for 2 minutes before excess fluid was blotted off. Except the sample in FIG. 9E, all samples were negatively stained. TEM micrographs were recorded using a JEOL 1200EX or JEOL JEM-1400Plus electron microscope operating at different kV.

### Scanning Electron Microscopy

Scanning Electron Microscopy (SEM) samples were prepared as described above for TEM on a 400-mesh copper grids, without uranyl acetate staining, and viewed using FBI Magellan™ 400L system.

### Kinetics of TPS4 Self-Assembly

Freshly prepared aliquots of 0.5, 1 or 2 mg/ml of TPS4 were placed in triplicates in a 96-well plate, 100 ul/well. Plate was placed at 37° C. in a Biotek Cytation 3 plate reader (Winooski, Vt., USA) and absorbance at 350 nm was measured every 2 minutes during 5 hours.

### Turbidity Analysis of TPS4 Degradation by Cathepsin B

Turbidity analysis for TPS4 solutions was conducted using triplicates of freshly prepared solutions as described above in a 96 well plate. Aliquots of 1, 2 or 4 mg/ml were allowed to assemble for 4-5 hours at room temperature. Next, 0.064, 0.128 or 0.255 μM of cathepsin B or acetate buffer vehicle were added, diluting the samples to 0.5, 1, and 2 mg/ml, respectively. The plate was placed at 37° C. in a Biotek Synergy HT plate reader (Winooski, Vt., USA) and absorbance was measured for 12 hours as described above. After 24 hours since enzyme addition, a color picture was taken and the turbidity was measured again.

### TPS4 Nanofibers Cytotoxicity

MDA-MB 231 human breast adenocarcinoma cells were cultured in Dulbecco's modified eagle's medium (DMEM) supplemented with 10% fetal bovine serum (FBS), 1% penicillin and 1% streptomycin in a humidified atmosphere of 5% CO₂ at 37° C. One day prior to treatment MDA-MB 231 cells (7×10³ cells/well) were cultured in a 96-well plate. The media of TPS4 that was assembled in DMEM was removed by centrifugation (14,000 rpm, 4° C.) and the nanofibers were resuspended in DMEM supplemented with 10% PBS, 1% penicillin and 1% streptomycin. Cells in triplicates were incubated with 0.5 mg/ml of TPS4 nanofibers (final concentration of 0.1% DMSO was kept constant) in growth medium at 37° C. After 24 or 48 hours of treatment, cell survival was determined by standard methylene blue assay.

### TPS4 Assembly in the Presence of Doxorubicin (Dox)

Assembly of TPS4 in the presence of Dox was achieved by premixing the peptide and the drug in DMSO followed by a dilution in DDW to final concentration of 1 mg/ml TPS4 and 5% w/w Dox. The assembly was preformed over-night in the dark.

### Preparation of Doxorubicin Particles

Doxorubicin.HCl (Sigma Aldrich), 1.06 mg, was dissolved in 2 μl dry DMSO. Then, 69 μl of 20 mM ammonium sulfate aqueous solution were added followed by a short sonication. Doxorubicin particles were precipitated by 10 minutes centrifugation at 14,000 rpm, 4° C. to obtain a red pellet and a light red supernatant. Supernatant was removed and re-precipitated by ammonium sulfate aqueous solution. Pellets were combined and resuspended in DDW to a final concentration of 10 mg/ml Doxorubicin particles (DP).

### DP-TPS4 Assembly

Assembly of TPS4 in the presence of DP was achieved by dilution while stirring of TPS4 in a solution of DP suspended in 20 mM ammonium sulfate. The assembly was preformed overnight in the dark. Next, access of Dox or DP were washed off by centrifugation (10 min, 14,000 rpm or 5 min, 8200 rpm, 4° C. respectively) and the resulting DP-TPS4 was resuspended in DDW to a final concentration of 1 mg/ml. For fluorescent characterization, 5 μl of each assembly were placed on a coverslip and allowed for complete dryness in the dark. Fluorescent images were taken at 60× magnification using an Olympus inverted fluorescent microscope IX51 equipped with a TritC filter (ex/em 535±50/610±75 nm).

### Determination of Dox Loading in DP-TPS4

Following DP-TPS4 assembly as described above, the supernatant of access Dox was collected by centrifugation (10 min, 14,000 rpm, 4° C.) and the resulting precipitation of DP-TPS4 was dissolved in DMSO. Dox fluorescence intensity in supernatant and precipitation was read at excitation/emission of 480/595 nm by a Cytation 3 plate reader and the amount of Dox was obtained from the calibration curve of Dox in 20 mM ammonium sulfate or DMSO respectively. The loading efficiency of DP in DP-TPS4 assembly was determined by the ratio of amount obtained from DP-TPS4 precipitation to the total amount obtained from supernatant and DP-TPS4 precipitation. Loading efficiency was determined in triplicate.

### Release of Dox by Cathepsin B

20% w/w DP or 5% w/w Dox in 1 mg/ml assembly (as described above) were placed in a dialysis unit (1 kDa molecular weight cut-off, Slide-A-Lyzer™ MINI Dialysis Devices, ThermoFisher Scientific). Next, cathepsin B was added to generate a final concentration of 0.5 mg/ml nanofibers assembly and 2 μM cathepsin B. The samples were placed at 37° C. and dialyzed while gently shaking, with dialysis replaced at indicated time points. At the end of the experiment, samples in dialysis units were collected and recovered. All collected samples were lyophilized and resuspended in DMSO and the fluorescence intensity of Dox was read at excitation/emission of 480/595 nm by a Cytation 3 plate reader. The amount of released Dox was obtained from the calibration curve of Dox in DMSO.

### Kinetics of Nanofibers Degradation by Tissue Lysates

Tissue lysates were prepared as previously described. Assembly of TPS4 in triplicates was performed for 4 hours in acetate buffer as described in FIG. 6B. Next, 100 μg of lysates from 4T1 tumor or muscles, or RIPA vehicle control (1% Tergitol-type NP-40 (nonyl phenoxypolyethoxylethanol), 0.1% SDS, 0.5% sodium deoxycholate) were added in acetate buffer. The absorbance was read at 350 nm after gentle shaking at 37° C. for indicated times using a BioTek plate reader.

1. A nanostructure comprising a plurality of peptides, at least a portion of the plurality of peptides comprises peptides having at least one pi-pi (π-π) interacting moiety and at least one protease interacting moiety, wherein the pi-pi interacting moiety and the at least one protease interacting moiety are different, wherein the at least one protease interacting moiety comprises at least one protease cleavable bond; and wherein the plurality of peptides is free of dipeptides.

2. The nanostructure according to claim 1, wherein each of said peptides in said plurality of peptides comprises between 3 and 10 amino acids.

3. The nanostructure according to claim 1, wherein at least a portion of the plurality of peptides has an amino acid sequence of the general formula X₂₍ₘ₎-X₃₍ₚ₎-X_{4(q)} (SEQ ID NO:1), wherein each of X₂, X₃ and X₄ is independently an amino acid and each of m, p and q is an integer independently selected from 1 to 3, wherein at least one of the amino acids or a combination of two or more amino acids constitutes a protease interacting moiety and at least one amino acid or a combination of two or more amino acids constitutes a pi-pi interacting moiety.

4. The nanostructure according to claim 1, wherein the at least one pi-pi interacting moiety comprises at least one aromatic moiety, being optionally an aromatic amino acid.

5. The nanostructure according to claim 1, being a cathepsin cleavable nanostructure, wherein the at least one protease interacting moiety comprises at least one cathepsin cleavable bond.

6. The nanostructure according to claim 1, being a nanofiber.

7. The nanostructure according to claim 1, wherein the at least one protease interacting moiety comprises at least one aromatic amino acid and at least one hydrophilic amino acid.

8. The nanostructure according to claim 7, wherein the hydrophilic amino acid is a positively charged amino acid selected from lysine, arginine and histidine.

9. The nanostructure according to claim 8, wherein the positively charged amino acid is lysine.

10. The nanostructure according to claim 1, wherein the at least one pi-pi interacting moiety is an aromatic amino acid selected from phenylalanine, tryptophan, tyrosine and histidine.

11. The nanostructure according to claim 10, wherein the aromatic amino acid is phenylalanine.

12. The nanostructure according to claim 1, wherein the protease is cathepsin B or cathepsin L.

13. The nanostructure according to claim 1, associated with at least one agent.

14. The nanostructure according to claim 13, wherein the at least one agent is associated to the surface of the nanostructure, encapsulated within the nanostructure or present inside a void in the nanostructure.

15. The nanostructure according to claim 1, wherein the peptide is selected from peptides having a sequence selected from the group consisting of SEQ ID NO:9, SEQ ID NO:10, SEQ ID NO:11, SEQ ID NO:12, SEQ ID NO:13, SEQ ID NO:14 and SEQ ID NO:15.

16. A drug delivery system comprising a nanostructure and at least one drug associated therewith, wherein the nanostructure comprises a plurality of peptides, at least a portion of the plurality of peptides comprises peptides having at least one pi-pi (π-π) interacting moiety and at least one protease interacting moiety, wherein the pi-pi interacting moiety and the at least one protease interacting moiety are different, and wherein the at least one protease interacting moiety comprises at least one protease cleavable bond.

17. A method selected from the group consisting of:
a. A method for treating a pathology associated with increased protease presence or for preventing onset of a pathology associated with increased protease presence in a subject, the method comprising administering to the subject a drug delivery system according to claim 16; and
b. A method for diagnosis of a pathology associated with increased protease presence in a subject, the method comprising administering to the subject a diagnostically effective amount of a drug delivery system according to claim 16 associated with an agent comprising an imaging moiety, and imaging the subject or a body region of the subject to thereby identify body regions in which said nanostructure has been localized.

18. The method according to claim 17 for treating a pathology associated with increased cathepsin presence or for preventing onset of a pathology associated with increased cathepsin presence in a subject.

19. The method according to claim 18, wherein the pathology is a proliferative disorder or an inflammatory disorder.

20. The method according to claim 17 for diagnosis of a pathology associated with increased cathepsin presence in a subject.


SEQUENCE LISTING (14 sequences)

SEQ ID NO: 1 - protein, 4 aa, Artificial Sequence
  peptide comprises between 3 to 9 amino acids and a combination of aromatic amino acids and  hydrophilic amino acids
  MISC_FEATURE (1)..(1): Xaa is equal to Xo, which is equal to any amino acid or absent
  MISC_FEATURE (2)..(2): Xaa is equal to X2, which is an aromatic amino acid or a hydrophilic amino acid
  MISC_FEATURE (3)..(3): Xaa is equal to X3, which is an aromatic amino acid or a hydrophilic amino acid
  MISC_FEATURE (4)..(4): Xaa is equal to X4, which is an aromatic amino acid or a hydrophilic amino acid, repeated q times, wherein q is an integer from 1 to 7
XXXX

SEQ ID NO: 2 - protein, 4 aa, Artificial Sequence
  peptide comprises between 4 to 12 amino acids and a combination of aromatic amino acids and  hydrophilic amino acids
  MISC_FEATURE (1)..(1): Xaa is equal to X1, which  is an aromatic amino acid or a hydrophilic amino acid,  repeated n times, wherein n is an integer from 1 to 3
  MISC_FEATURE (2)..(2): Xaa is equal to X2, which  is an aromatic amino acid or a hydrophilic amino acid
  MISC_FEATURE (3)..(3): Xaa is equal to X3, which  is an aromatic amino acid or a hydrophilic amino acid
  MISC_FEATURE (4)..(4): Xaa is equal to X4, which  is an aromatic amino acid or a hydrophilic amino acid, repeated q times, wherein q is integer from 1 to 7
XXXX

SEQ ID NO: 3 - protein, 4 aa, Artificial Sequence
  peptide comprises a combination of aromatic amino acids and hydrophilic amino acids
  MISC_FEATURE (1)..(1): Xaa is equal to X0 which is any amino acid or absent
  MISC_FEATURE (2)..(2): Xaa is equal to X2, which is an aromatic amino acid or a hydrophilic amino acid, wherein if X0 is absent, then X2 is substituted by R1 which is an N-capping group
  MISC_FEATURE (3)..(3): Xaa is equal to X3, which is an aromatic amino acid or a hydrophilic amino acid
  MISC_FEATURE (4)..(4): Xaa is equal to X4 which is an aromatic amino acid or a hydrophilic amino acid, repeated q times, wherein q is an integer from 1 to 7,  wherein  X4 is substituted by R2 being a C-capping group
XXXX

SEQ ID NO: 4 - protein, 4 aa, Artificial Sequence
  peptide comprises between 4 to 12 amino acids and a combination of aromatic amino acids and  hydrophilic amino acids
  MISC_FEATURE (1)..(1): Xaa is equal to X1, wherein X1 is an aromatic amino acid or a hydrophilic amino acid, repeated n times, wherein n is an integer from 1 to 3 and X1 is substituted by R1, which is an N-capping group
  MISC_FEATURE (2)..(2): Xaa is equal to X2, which is an aromatic amino acid or a hydrophilic amino acid
  MISC_FEATURE (3)..(3): Xaa is equal to X3, which is an aromatic amino acid or a hydrophilic amino acid
  MISC_FEATURE (4)..(4): Xaa is equal to X4, wherein  X4 is an aromatic amino acid or a hydrophilic amino acid, repeated q  times, wherein q is an integer from 1 to 7, wherein X4 is substituted by R2 being a C-capping group
XXXX

SEQ ID NO: 5 - protein, 4 aa, Artificial Sequence
  peptide comprises a combination of aromatic amino acids and hydrophilic amino acids
  MISC_FEATURE (1)..(1): Xaa is equal to Xo, which is any amino acid, or absent
  MISC_FEATURE (2)..(2): Xaa is equal to X2 being an aromatic amino acid or a hydrophilic amino acid, wherein if X0 is absent, then X2 is substituted by R1 being N-capping group
  MISC_FEATURE (3)..(3): Xaa is equal to X3, which is an aromatic amino acid or a hydrophilic amino acid
  MISC_FEATURE (4)..(4): Xaa is equal to X4,which  is an aromatic amino acid or a hydrophilic amino acid, repeated q times wherein q is an integer from 1 to 7
XXXX

SEQ ID NO: 6 - protein, 4 aa, Artificial Sequence
  peptide comprises a combination of aromatic amino acids and hydrophilic amino acids
  MISC_FEATURE (1)..(1): Xaa is equal to Xo, which is any amino acid, or absent
  MISC_FEATURE (2)..(2): Xaa is equal to X2, which is an aromatic amino acid or a hydrophilic amino acid
  MISC_FEATURE (3)..(3): Xaa is equal to X3 , which  is an aromatic amino acid or a hydrophilic amino acid
  MISC_FEATURE (4)..(4): Xaa  is equal to X4 is an aromatic amino acid or a hydrophilic amino acid, repeated q times, wherein q is an integer from 1 to 7, wherein X4 is substituted by R2 being a C-capping group
XXXX

SEQ ID NO: 7 - protein, 4 aa, Artificial Sequence
  peptide comprises between 4 to 12 amino acids and a combination of aromatic amino acids and  hydrophilic amino acids
  MISC_FEATURE (1)..(1): Xaa is equal to  X1 being  an aromatic amino acid or a hydrophilic amino acid, repeated n times, wherein n is an integer from 1 to 3, wherein X1 is substituted by  R1 being N-capping group
  MISC_FEATURE (2)..(2): Xaa is equal to X2, which is an aromatic amino acid or a hydrophilic amino acid
  MISC_FEATURE (3)..(3): Xaa is equal to X3, which is an aromatic amino acid or a hydrophilic amino acid
  MISC_FEATURE (4)..(4): Xaa is equal to X4, which is an aromatic amino acid or a hydrophilic amino acid, repeated q times, wherein q is an integer from 1 to 7
XXXX

SEQ ID NO: 8 - protein, 4 aa, Artificial Sequence
  peptide comprises between 4 to 12 amino acids and a combination of aromatic amino acids and hydrophilic amino acids
  MISC_FEATURE (1)..(1): Xaa is equal to X1, which is an aromatic amino acid or a hydrophilic amino acid, repeated n times, wherein n is an integer from 1 to 3
  MISC_FEATURE (2)..(2): Xaa is equal to X2, which is  an aromatic amino acid or a hydrophilic amino acid
  MISC_FEATURE (3)..(3): Xaa is equal to X3, which is an aromatic amino acid or a hydrophilic amino acid
  MISC_FEATURE (4)..(4): Xaa is equal to X4 which is an aromatic amino acid or a hydrophilic amino acid, repeated q times, wherein q is an integer from 1 to 7 wherein X4 is substituted by group R2 being C-Capping group
XXXX

SEQ ID NO: 9 - protein, 4 aa, Artificial Sequence
  peptide
FFKF

SEQ ID NO: 10 - protein, 4 aa, Artificial Sequence
  peptide
  MISC_FEATURE (1)..(1): Xaa is equal to Phe (F) substituted with acetyl (Ac)
XFKF

SEQ ID NO: 11 - protein, 4 aa, Artificial Sequence
  peptide
  MISC_FEATURE (1)..(1): Xaa is equal to  Phe (F) substituted with Fluorenylmethyloxycarbonyl (Fmoc)
XFKF

SEQ ID NO: 12 - protein, 4 aa, Artificial Sequence
  peptide
  MISC_FEATURE (1)..(1): Xaa is equal to Phe (F) substituted with benzyloxycarbonyl (Cbz)
XFKF

SEQ ID NO: 13 - protein, 4 aa, Artificial Sequence
  peptide
  MISC_FEATURE (1)..(1): Xaa is equal to Phe (F) substituted with Fluorenylmethyloxycarbonyl (Fmoc)
  MISC_FEATURE (4)..(4): Xaa is equal to Phe (F) substituted with an amide (NH2)
XFKX

SEQ ID NO: 14 - protein, 4 aa, Artificial Sequence
  peptide
  MISC_FEATURE (1)..(1): Xaa is equal to Phe (F) substituted with benzyloxycarbonyl (Cbz)
  MISC_FEATURE (4)..(4): Xaa is equal to Phe (F) substituted with an amide (NH2)
XFKX